(19) United States
(12) Patent Application Publication
(10) Pub. No.: US 2025/0314642 A1
(43) Pub. Date: Oct. 9, 2025

(54) METHODS FOR DEPLETION AND ENRICHMENT

(71) Applicant: Veravas, Inc., Austin, TX (US)

(72) Inventors: Joshua Caine Soldo, Prior Lake, MN (US); Scott Douglas Bergmann, River Falls, WI (US); Carmen Leah Wiley, Spokane, WA (US)

(21) Appl. No.: 19/185,839

(22) Filed: Apr. 22, 2025

Related U.S. Application Data
(63) Continuation of application No. 17/160,196, filed on Jan. 27, 2021.
Application No. 17/160,196 is a continuation of application No. PCT/US2019/043711, filed on Jul. 26, 2019.
(60) Provisional application No. 62/711,391, filed on Jul. 27, 2018.

(51) Int. Cl. G01N 33/53; B01D 15/38; B01D 21/26; B03C 1/03; G01N 33/543; G01N 33/68

(52) U.S. Cl. CPC G01N 33/5306; B01D 15/3809; B01D 15/3823; B01D 21/262; B03C 1/03; G01N 33/54326; G01N 33/6848; B03C 2201/18; G01N 2560/00

(57) ABSTRACT

The present invention is directed to methods for using particles (e.g, microparticulate, nanoparticulate; magnetic, non-magnetic) comprising surfaces comprising capture moieties as described herein, to remove an interference as described herein, or enrich biomarkers, prior to a diagnostic test.

### CROSS-REFERENCE TO RELATED APPLICATIONS

This application claims the benefit of priority to U.S. Provisional Patent Application No. 62/711,391 filed Jul. 27, 2018. The entire content of the aforementioned application is incorporated herein by reference for all purposes.

### FIELD OF THE INVENTION

The present invention relates to methods for using particles (e.g, microparticulate, nanoparticulate; magnetic, non-magnetic) comprising surfaces comprising capture moieties as described herein to isolate biomarkers for subsequent depletion or enrichment applications prior to a diagnostic test.

### BACKGROUND OF THE INVENTION

Laboratory testing plays a critical role in health assessment, health care, and ultimately the public's health, and affects persons in every life stage. Almost everyone will experience having one or more laboratory tests conducted during their lifetime. A n estimated 7 to 10 billion laboratory tests are performed each year in the United States alone, and laboratory test results influence approximately 70% of medical decisions.

In addition, since the Centers for Medicare and Medicaid Services (CMS) on Jan. 1, 2018 implemented the new Clinical Laboratory Fee Schedule (CLFS) as required by the Protecting Access to Medicare Act (PAMA), PAMA is reducing lab testing reimbursement. It is even more critical lab results are accurate the first time and troubleshooting efforts are reduced or take less time and do not impact lab workflow.

Interference is a substance present in a patient specimen that can alter the correct value of the result of a diagnostic test, e.g., by interfering with antibody binding, or that can increase or decrease assay signal by bridging, steric hindrance, or autoantibody mechanisms. While it is known that immunoassays are susceptible to interference, the clinical laboratory may still report erroneous results if such results are not recognized and flagged by the instrument (analyser) or laboratory, or if the physician does not notify the laboratory that the patient result does not fit the clinical picture. Erroneous results can occur unexpectedly with any specimen without the practical means to identify upfront such specimens likely to cause problems. The consequence of such interference is that erroneous results can result in false negatives and false positive test results, that can impact patient care, and can lead to unnecessary invasive, diagnostic or therapeutic procedures, or failure to treat a patient.

Notwithstanding the complications arising from interference, biomarker screening and diagnostic testing can be difficult, for example because of their low presence or abundance in a biological sample.

Thus, while biomarkers found in the body can be used to detect, predict, or manage diseases, many are found in too low an abundance to be detected today using commercially available tests. There is an unmet clinical need for new diagnostic technology that prepares clinical samples to improve testing accuracy, measure hard to find biomarkers, reduce costs, and ultimately save lives.

Biotin, also known as vitamin B7, is a water-soluble B vitamin often found in multivitamins and over the counter health and beauty supplements. In vitro laboratory diagnostics tests that employ streptavidin-biotin binding mechanisms have the potential to be affected by high circulating biotin concentrations. Biotin can be attached through covalent bond to a variety of targets—from large antibodies to steroid hormones—with minimal effect on their specific non-covalent binding with avidin, streptavidin, or NeutrAvidin proteins. Therefore, biotin has been frequently used in the detection systems of immunoassays of different forms.

Immunoassays are generally categorized as either sandwich immunoassays (non-competitive) or competitive inhibition immunoassays. In general, streptavidin-biotin binding is used during assay incubation to couple biotinylated antibodies in sandwich immunoassays, or biotinylated antigens in competitive immunoassays, to streptavidin-coated surfaces. When a biological specimen contains excess biotin, the biotin competes with the biotinylated antibodies or antigens for binding to the streptavidin-coated surfaces, resulting in reduced capture of the biotinylated antibodies or antigens. Excess biotin produces falsely low results in sandwich immunoassays because the assay signal is directly proportional to the analyte concentration. Excess biotin in competitive immunoassays causes falsely elevated results because the assay signal is inversely proportional to the analyte concentration.

Normal circulating concentrations of biotin derived from the diet and normal metabolism are too low (<1 ng/mL) to interfere with biotinylated immunoassays. However, ingestion of high-dose biotin supplements (e.g., 5 mg or higher) can result in significantly elevated blood concentrations that can interfere with commonly used biotinylated immunoassays. In certain medical conditions, extremely high biotin doses (e.g., 100 mg or higher) can result in serum or plasma biotin levels of >1000 ng/mL.

Biotin in blood or other samples taken from patients who are ingesting high levels of biotin can cause falsely high or falsely low results in biotin-based immunoassays, depending on the design of the assay. Incorrect test results may lead to inappropriate patient management as well as misdiagnosis.

Biotin interference thresholds differ widely among assays, even on a single platform. Tests with biotin interference thresholds <51 ng/mL are considered high risk tests, or vulnerable immunometric and competitive methods.

There is therefore a clinical need for simple, inexpensive, automatable and effective solutions to eliminate or minimize sample interference and enrich biomarker concentration prior to diagnostic testing without impacting laboratory workflow and turnaround time.

### SUMMARY OF THE INVENTION

Described herein are methods for the simple, efficient and cost-effective conditioning of biological samples to manage and mitigate a multitude of known sample-specific interferences that can lead to erroneous test results and increased risk to patient safety, such as heterophilic antibodies in patients who have been treated with monoclonal mouse antibodies or have received them for diagnostic purposes. The methods described herein can also manage and mitigate sample-specific interferences that arise from biotin that can come from over the counter (OTC) supplements, multivitamins and herbal remedies taken by consumers for health & beauty and weight loss or therapeutically, e.g., for the treatment of multiple sclerosis.

Also described herein are methods for enriching or increasing the concentration of a biomarker in a biological sample.

In an aspect, provided herein is a method for isolating a biomarker from a biological sample, the method comprising: a) combining the sample with a particle comprising a capture moiety to provide a mixture; and b) mixing the mixture to provide particle complexes to the biomarker; thereby isolating the biomarker from the biological sample.

In an aspect, provided herein is a method for removing an interference from a biological sample, the method comprising: a) combining the sample with a particle comprising a capture moiety to provide a mixture; b) mixing the mixture to provide particle complexes to the interference; and c) removing or eliminating the particle complexes to provide a depleted solution; thereby decreasing or reducing the amount (e.g., mass, molarity, concentration) of the interference.

### BRIEF DESCRIPTION OF THE DRAWINGS

FIG. 1 depicts a scheme for confirmation and disqualification assays based on removal (or depletion) of interferences from a biological sample by particles described herein.

FIG. 2 depicts a scheme for depletion assays based on removal (or depletion) of interferences from a biological sample by lyophilized particles described herein.

FIG. 3 depicts a scheme for depletion assays based on removal (or depletion) of interferences from a biological sample by magnetized pipette tips described herein.

FIG. 4 is a graph showing biotin concentration over time after biotin ingestion.

FIG. 5 is a graph showing biotin depletion.

FIG. 6 is a graph showing biotin depletion.

FIG. 7 is a graph showing biotin concentration over time after biotin ingestion.

FIG. 8 is a graph showing biotin concentration after ingestion of different biotin doses.

FIG. 9 is a graph showing biotin depletion.

FIG. 10 is a graph showing PTH concentration.

### DETAILED DESCRIPTION OF THE INVENTION

Described herein are methods for depleting or enriching a biological sample, the method comprising combining particles as described herein with a biological sample as described herein.

In an aspect, provided herein is a method for isolating a biomarker from a biological sample, the method comprising: a) combining the sample with a particle comprising a capture moiety to provide a mixture; and b) mixing the mixture to provide particle complexes to the biomarker; thereby isolating the biomarker from the biological sample.

In some embodiments, the method further comprises subjecting the particle complexes to diagnostic testing.

In an aspect, provided herein is a method for removing an interference from a biological sample, the method comprising: a) combining the sample with a particle comprising a capture moiety to provide a mixture; b) mixing the mixture to provide particle complexes to the interference; and c) removing or eliminating the particle complexes to provide a depleted solution; thereby decreasing or reducing the amount (e.g., mass, molarity, concentration) of the interference.

In some embodiments, the method further comprises subjecting the depleted solution to characterization (e.g., a diagnostic test).

In some embodiments, the particle is provided as a lyophized product (e.g., a LyoSphere™ (BIOLY PH LLC)).

In an aspect, provided herein is a method for increasing the accuracy of a diagnostic test, the method comprising: a) combining a biological sample with a particle comprising a capture moiety to provide a mixture; b) mixing the mixture to provide particle complexes to the interference; c) removing or eliminating the particle complexes to provide a depleted solution; and d) subjecting the depleted solution to the diagnostic test; thereby increasing the accuracy of the diagnostic test.

In some embodiments, at least 1%, 3%, 5%, 10%, 20%, 30%, 40%, 50%, 60%, 70%, 80%, 90%, 99% of the interference is removed in comparison to a biological sample not subjected to the method. In some embodiments, a sufficient amount of interference is removed to provide less than 100 ppm interference in the biological sample. In some embodiments, a sufficient amount of interference is removed to provide a less than detectable amount of the interference in a diagnostic test.

In some embodiments, the capture moiety is a human anti-animal antibody (e.g., mouse IgG, sheep IgG, goat IgG, rabbit IgG, cow IgG, pig IgG, horse IgG). In some embodiments, the capture moiety is a heterophilic antibody (e.g., FR (Fc-specific), Fab, F(ab)′2, polymerized IgG (type 1, 2a, 2b IgG and IgG fragments, serum components). In some embodiments, the capture moiety is an assay specific binder (e.g., biotin, fluorescein, anti-fluorescein poly/M ab, anti-biotin poly/M ab, streptavidin, neutravidin). In some embodiments, the capture moiety is an assay specific signal molecule (e.g., HRP, ALP, acridinium ester, isoluminol/luminol, ruthenium, N-(4-aminobutyl)-N-ethylisoluminol (ABEI)/cyclic ABEI). In some embodiments, the capture moiety is an assay specific blocker (e.g., BSA, fish skin gelatin, casein, ovalbumin, PVP, PVA). In some embodiments, the capture moiety is an assay specific conjugate linker (e.g., LC, LC-LC, PEO4, PEO16). In some embodiments, the capture moiety is an antigen autoantibody (e.g., free T3, free T4). In some embodiments, the capture moiety is a protein autoantibody (e.g., MTSH, TnI, TnT, non-cardiac TnT (skeletal muscle disease)). In some embodiments, the capture moiety is a chemiluminescent substrate (e.g., luminol, isoluminol, isoluminol derivatives, ABEI, ABEI derivatives, ruthenium, acridinium ester) or fluorescent label (e.g., fluorescein or other fluorophores and dyes). In some embodiments, the capture moiety is streptavidin, neutravidin, avidin, polyA, polyDT, aptamers, antibodies, Fab, F(ab′)2, antibody fragments, recombinant proteins, enzymes, proteins, biomolecules, or polymers. In some embodiments, the capture moiety is biotin, fluorescein, PolyDT, PolyA, antigen, etc.

In some embodiments, the removing or eliminating is a separation. In some embodiments, the separation comprises physical separation. In some embodiments, the separation comprises magnetic separation. In some embodiments, the magnet for the magnetic separation is a multiple magnet device containing 2 to 12 magnets in a rack designed to hold 1 to 12 sample preparation tubes on a large pipetting machine. Examples of such pipetting machines include, but are not limited to, those built by Hamilton or Tecan. In some embodiments, the magnet for the magnetic separation is a multiple magnet device containing 96 or 384 magnets designed to provide magnetization to a 96 well or 384 well microtiter plate. In some embodiments, the separation comprises chemical separation. In some embodiments, the removing or eliminating comprises centrifugation at 1000×g or greater for at least 1 minute, 2 minutes, 3 minutes, 4 minutes, or 5 minutes to provide a pellet and a supernatant; and removing the supernatant. In some embodiments, the removing or eliminating comprises filtration (e.g., through a filter. In some embodiments, the filter has porosity or molecular weight cut-off (MWCO) sufficiently smaller than the diameter of the particle (e.g., nanoparticle, microparticle). In some embodiments, the filtration is by gravity, vacuum, or centrifuge. In some embodiments, the removing or eliminating comprises magnetization. In some embodiments, the magnetization occurs using a strong magnet (e.g., a neodymium magnet); to provide a pellet and a supernatant. In some embodiments, the magnet is in the centrifuge rotor. In some embodiments, the magnet is a magnet within a disposable pipette tip, cover or sheath.

In an aspect, provided herein is a method for isolating a biomarker from a biological sample, the method comprising: a) combining the sample with a particle comprising a capture moiety to provide a mixture; b) mixing the mixture to provide particle complexes comprising the biomarker; c) removing the particle complexes from the mixture; and d) adding to the mixture a cleavage reagent or releasing agent to provide an isolate comprising the biomarker; thereby isolating the biomarker from the biological sample. In some embodiments, the method for isolating a biomarker from a biological sample is performed prior to performing a diagnostic test on the biological sample.

In an aspect, provided herein is a method for determining whether a biomarker is present in a biological sample, the method comprising: a) combining the sample with a capture moiety to provide a mixture; b) combining the mixture with a particle comprising the capture moiety to provide a tertiary complex; c) removing the tertiary complex from the mixture to provide an isolate; and d) determining whether an indicator for the tertiary complex is present in the isolate; thereby determining whether the biomarker is present in a biological sample.

In an aspect, provided herein is a method for determining whether a biomarker is present in a biological sample, the method comprising: a) combining the sample with a particle comprising a capture moiety to provide a mixture; b) mixing the mixture to provide a particle complex to the interference; c) removing or eliminating the particle complexes to provide a depleted solution; d) combining the depleted solution with a second particle comprising a second capture moiety to provide a second mixture; e) mixing the second mixture to provide a second particle complex comprising the biomarker; f) removing the second particle complex from the second mixture; and g) adding to the second mixture a cleavage reagent or releasing agent to provide an isolate comprising the biomarker; thereby isolating the biomarker from the biological sample.

In some embodiments, the method further comprises washing the particle complex with a diluent.

In some embodiments, the cleavage reagent is a disulfide bond reducing reagent.

In some embodiments, the method further comprises performing a diagnostic test on the biomarker.

In an aspect, provided herein is a method for enriching an amount of a biomarker in a sample, the method comprising: a) adding to the sample a particle comprising a capture moiety to provide a mixture; b) mixing the mixture to provide a particle complex; c) separating the particle complex to provide a pellet and a supernatant; e) removing the supernatant from the pellet; f) washing the pellet with a diluent; and g) eluting the biomarker from the pellet to provide an enriched sample; thereby enriching the amount of a biomarker in the sample. In some embodiments, the method for enriching a biomarker from a biological sample is performed prior to performing a diagnostic test on the biological sample.

In some embodiments, the biomarker is an indicator of traumatic brain injury (TBI). In some embodiments, the biomarker is s-100β, glial fibrillary acidic protein (GFAP), neuron-specific enolase (NSE), neurofilament light chain (NFL), cleaved tau protein (C-tau), and ubiquitin C-terminal hydrolase-L1 (UCH-L1). In some embodiments, the biomarker is an indicator of Alzheimer's Disease (AD). In some embodiments, the biomarker is amyloid beta, BACE1, soluble Aβ precursor protein (sAPP). In some embodiments, the biomarker is an indicator of a sexually transmitted disease (STD). In some embodiments, the STD is Chlamydia, Gonorrhea, Syphilis, Trichomonas, HPV, Herpes, Hepatitis B, Hepatitis C, HIV. In some embodiments, the biomarker is an indicator of bacterial infection. In some embodiments, the biomarker is capture moiety for a bacterium. In some embodiments, the biomarker is cleaved from the complex by a cleavage reagent. In some embodiments, the presence of biomarker is determined by MALDI-MS. In some embodiments, the presence of biomarker is determined by a molecular diagnostic method. In some embodiments, the presence of biomarker is determined by an immunoassay.

In some embodiments, the interference is fibrinogen and the removing or eliminating is separation, such as a physical separation by centrifugation, wherein the particle complexes are entrapped in a clot.

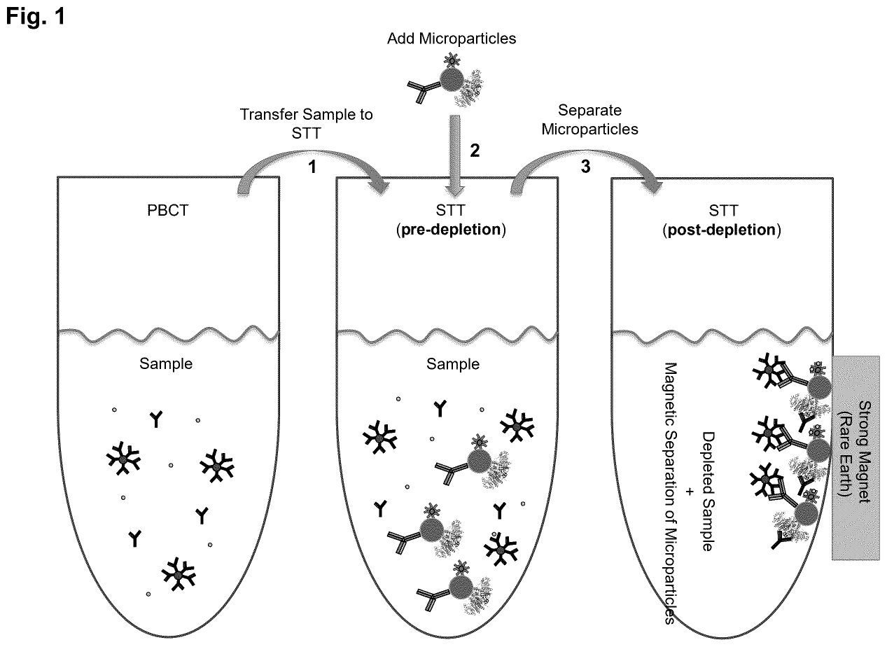

Turning to FIG. 1, a scheme is shown for confirmation and disqualification assays based on removal (or depletion) of interferences from a biological sample by particles described herein.

A biological sample is aspirated from a primary blood collection tube (PBCT) and dispensed into the secondary transfer tube (STT). Particles described herein, e.g., particles comprising surfaces comprising capture moieties for free biotin and/or heterophilic antibodies are added to the STT to bind and deplete sample interferences.

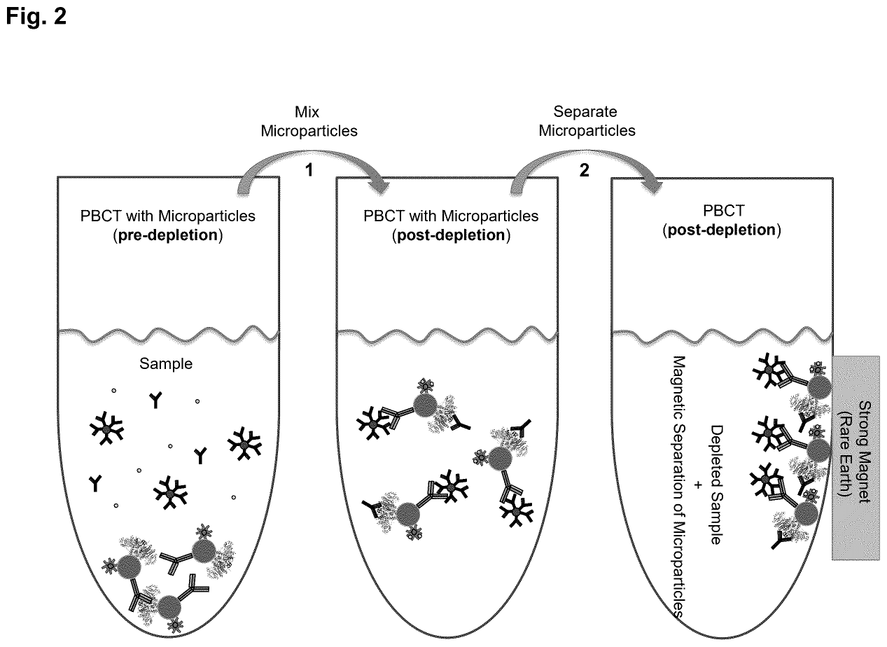

In FIG. 2, a scheme is shown for depletion assays based on removal (or depletion) of interferences from a biological sample by lyophilized particles described herein. A PBCT comprising lyophilized particles (e.g., particles as described herein) receive a biological sample, resulting in the resuspension and dispersement of particles with the biological sample.

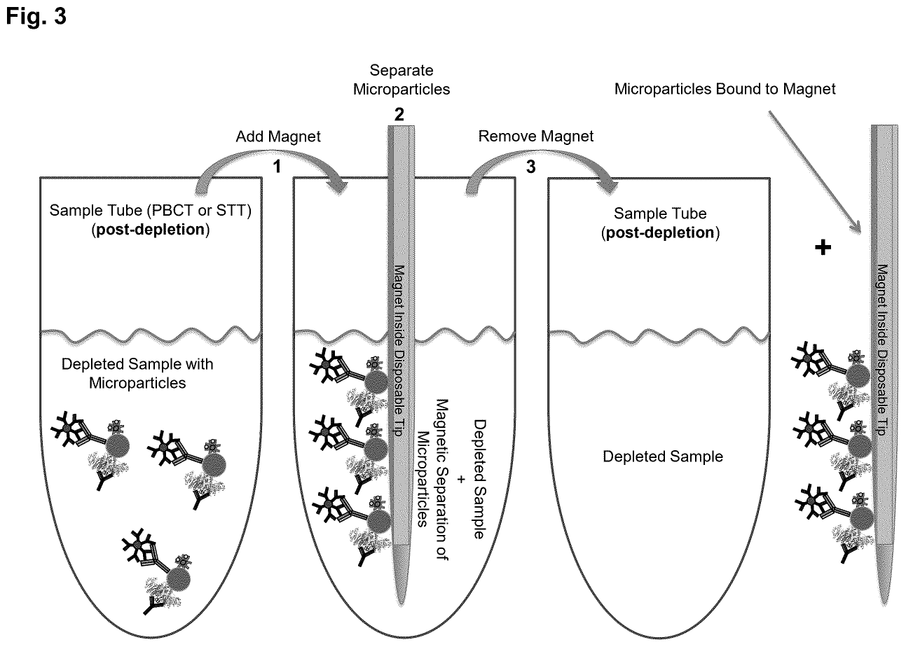

In FIG. 3, a scheme is shown for depletion assays based on removal (or depletion) of interferences from a biological sample by magnetized pipette tips described herein. A pipette tip comprising a magnet is added to a biological sample to remove from the biological sample an interference as described herein or biomarker as described herein.

### Method of Separation

Particles described herein can be added to a collection device such as a primary blood collection tube, 24-hr urine collection device, a urine collection device, a saliva collection tube, a stool collection device, a seminal fluid collection device, a blood collection bag, or any sample collection tube or device, prior to the addition of the biological sample.

Particles described herein can also be added to a sample after collection of the sample into a collection device, or after the transfer of the sample from a primary collection device into a storage or transfer device such as a plastic or glass tube, vial, bottle, beaker, flask, bag, can, microtiter plate, ELISA plate, 96-well plate, 384-well plate 1536 well plate, cuvette, reaction module, reservoir, or any container suitable to hold, store or process a liquid sample.

In some embodiments, the particles described herein are added to a collection device comprising a biological sample. In some embodiments, the particles described herein are added to a collection device prior to addition of a biological sample.

In an aspect, described herein is a device for releasing particles comprising a collection device as described herein comprising a biological sample (i.e. screw cap which triggers release mechanism) such as on a urine collection device. For example, the device is a tube equipped with a screw cap that releases the particles described herein upon closure of the screw cap.

In an aspect, described herein is a device comprising a chemical release of particles to a container comprising a biological sample (i.e. encapsulated composition or composition that dissolves in solution at a defined rate or point in time). In some embodiments, the devices described herein are configured to delay the addition of particles described herein, for example to provide pre-treatment of sample prior to diagnostic testing.

In some embodiments, the sample described herein can be pre-treated with a chemical, protein, blocker, surfactant or combination thereof prior to addition of the particles described herein for example to adjust pH, deplete or compete for sample specific interferences, and/or manage matrix specific challenges prior to the nanoparticles being added, introduced, dispersed or mixed in the sample to improve the specificity and binding kinetics of the nanoparticles to the target biomarker(s). The delayed addition of the nanoparticles to the sample after sample pre-treatment can be controlled physically by adding the nanoparticles to the sample after sample pre-treatment. The nanoparticles can also be present in the sample during the sample pre-treatment if the nanoparticles are encapsulated, shielded or protected by a chemical, polymer or sugar shell, coating, or polymerization such that the chemical, polymer or sugar needs to dissolve before the nanoparticles can be released, added, dispersed or mixed in the sample. The delayed release of nanoparticles can use chemistry known to someone skilled in the art such as used today in delayed drug release technology.

Methods of M agnetic Separation of Particles

In one aspect, provided herein is a method for removing an interference from a biological sample (e.g., prior to a diagnostic test), or to isolate or separate magnetic particle (e.g., within a primary blood collection tube, custom sample collection device, secondary transfer tube or custom sample device). For example, a magnet-based device will quickly (less than 2 minutes; preferably less than 30 seconds) isolate the magnetic nanoparticles to the side(s) and/or bottom to form an essentially particle-free supernatant. The particle-free supernatant can be subsequently aspirated without disrupting the pellet comprising the particles and dispensed into a separate transfer tube for diagnostic testing. In some embodiments, the pellet is isolated or subjected to diagnostic testing.

### Devices for the M Agnetic Separation of Particles

Provided herein are devices comprising particles as described herein that can be used in the methods described herein to remove or deplete biomarkers, for example for diagnostic testing. In some embodiments, the devices comprise a physical mechanism to delay combination of the particles described herein with the samples described herein. In some embodiments, the devices described herein comprise a timed release mechanism to delay combination of the particles described herein with the samples described herein.

Magnetic Tube Holder. A custom magnetic tube holder, or a custom magnetic tube holder that can be removed from its stand, that can be inserted inside a sample rack for subsequent diagnostic testing of the particle-free supernatant. The custom magnetic tube holder can be designed to have physical openings or clear/transparent plastic (where magnets or the magnet array are not present) in its design where the sample tube barcode can still be detected and read by the analyzer, or where indices tests such as lipemia, hemolysis, cellular debris/clot detection, level sensing, etc. can still be performed by the analyzer. The sample tube could be a custom sample tube designed to have notches, or tongue and groove design, to only fit in the custom magnetic tube holder in a specific orientation to ensure the magnetic tube holder openings (space) or clear/transparent plastic allows the analyzer to see and read the barcode and/or perform indices tests such as lipemia, hemolysis, cellular debris/clot detection, level sensing, etc.

In some embodiments, the use of magnet(s) that can be attached to a sample rack via an adhesive, Velcro, or other method. Once the sample tube containing magnetic nanoparticles is inserted into the sample rack position(s) with magnet(s), the magnetic nanoparticles will quickly separate to the side(s) and/or bottom of the sample tube to form an essentially particle-free sample supernatant for diagnostic testing by the sample-rack specific testing platform or analyzer.

The sample rack itself as a custom magnetic sample rack compatible with a given analyzer (e.g., specific for the Abbott ARCHITECT, Siemens ADVIA Centaur XP, Roche cobas e411/e601/602/e801, Beckman Coulter Access 2/Dxl 400/Dxl 800, DiaSorin LIAISON/LIAISON XL, etc.). For example, every tube position in the rack will have an array of magnets designed to quickly separate the magnetic nanoparticles to the side(s) and/or bottom of the sample tubes to form essentially particle-free sample supernatants for diagnostic testing.

In an aspect, provided herein is a device (e.g., separation device) comprising a holder (e.g., a test tube holder) for a rack of test tubes, wherein the holder comprises a magnet.

Disposable Pipette Tip. In an aspect, the device is a disposable pipette tip comprising a custom magnet inserted inside the disposable tip to quickly isolate the magnetic nanoparticles to the surface of the pipette tip to form an essentially particle-free sample supernatant. The disposable pipette tip with custom magnet can subsequently be removed from the sample without disrupting the pellet comprising the particles. The disposable tip comprising particles can be discarded if there is no need to measure or characterize what the particles captured (i.e. interference depletion), or it can be inserted into a new tube for isolation and characterization of the particles in a subsequent diagnostic test (i.e., enrichment). For example, the disposable tip with particles can be inserted into a secondary transfer tube containing a buffer. If the magnet is removed from the tip, or if the magnet is turned off (e.g., electromagnet) the particles are free to disperse into the buffer.

In an aspect, provide herein is a device comprising a disposable pipette tip, wherein the tip comprises a magnet.

### Methods of Physical Separation of Particles

In one aspect, described herein are methods for removing particles described herein by physical force (e.g., gravitational force). In some embodiments, the particles described herein are separated, isolated, or removed (e.g., by centrifugation) from a biological sample by physical force. In some embodiments, the methods are used prior to application of diagnostic test methods described herein, for example, within a primary blood collection tube, custom sample collection device, secondary transfer tube or custom sample device. In some embodiments, the method for removing particles is filtration.

For example, magnetic nanoparticles specific for fibronectin and/or other clotting factors or off the clot components/constituents, cellular debris (i.e. red blood cell membrane specific) for the subsequent capture or binding of the “clot” (in serum) and/or capture or binding of cellular debris (in serum or plasma) enhance centrifugation speed and efficiency (shorter spin times to improve lab efficiency, workflow and throughput) by integration of strong magnets or magnetic technology in the centrifuge rotor and/or tube holders. This combination of RCF or Gs from centrifugation with magnetic separation of the magnetic nanoparticle complex (i.e. clot+magnetic beads, cellular debris+magnetic beads) enable much quicker and more efficient separation and supernatant formation on the side or bottom of the sample tube to clarify the sample for subsequent analysis. For example, this centrifugation step in most laboratories is 4 minutes or greater, and may be reduced to 2 minutes or less (preferable 1 min or less) by combining centrifugation with magnetic separation/isolation of the magnetic nanoparticle clot/cellular debris complexes.

Moreover, if the nanoparticles or plurality of magnetic nanoparticles are also specific for one or more different sample interference mechanisms such as 1, 5, 10, 20, 30, or more different interference mechanisms, these interferences, if present, would be captured by the nanoparticles and depleted from the sample after physical separation from centrifugation, or by the combination of centrifugation and magnetic separation described above.

While these magnetic nanoparticles do not need to also have specificity to the clot or cellular debris to be isolated via centrifugation or the combination of centrifugation and magnetic separation in the centrifuge, their surface could be co-coated or immobilized with more than one antibody and/or antigen where one or more antibodies would be specific for the clot and/or cellular debris, while the other antibody(s) and/or antigen would be specific to the sample interference. In this regard, the nanoparticles would specifically bind to both sample interference as well as the clot and/or cellular debris for subsequent physical separation or isolation via centrifugation or the combination of centrifugation and magnetic separation.

The use of nanoparticles specific for the clot and/or cellular debris increase clotting rate of speed by specific binding by the magnetic nanoparticles and pulling everything to a magnetic for magnetic separation and isolation. This bead-based pellet formed by the magnetic field and strength also accelerates the clot formation based on forced proximity of the clot or specifically captured clotting factors by the nanoparticles and subsequently the magnet.

### Methods of Chemical Separation of Particles

In some embodiments, the particles described herein are separated, isolated, or removed from a biological sample by chemical separation methods. In some embodiments, the chemical separation methods are used prior to application of diagnostic test methods, for example, within a primary blood collection tube, custom sample collection device, secondary transfer tube or custom sample device.

In one aspect, provided is a method for chemical separation of particles, the method comprising providing one or more of a salt, solvent, polymer, or detergent.

In some embodiments, the chemical separation methods, e.g., liquid-liquid phase separation will partition particles into a Phase A, and the nanoparticle free sample will be portioned into a Phase B where Phase B is tested. The agents for liquid-liquid phase separation (chemical phase separation) can be by salts, soluble polymers and detergents.

For example, liquid-liquid phase separation can occur by adding a non-polar solvent such as hexane to the polar aqueous sample where the particles partition into the non-polar phase leaving a nanoparticle-free aqueous phase for testing by a diagnostic test as described herein. In some embodiments, the method of separation described herein provide nanoparticles in the organic phase. In some embodiments, the method of separation described herein provide nanoparticles in the aqueous phase.

A method for isolating particles in a biological sample, the method comprising providing to the particles and biological sample a nonpolar solvent and an aqueous polar solvent to provide a nonpolar solvent layer and a polar solvent layer, removing a nonpolar solvent layer comprising the nonpolar solvent, and isolating the aqueous polar solvent comprising the particles, thereby isolating the particles.

Sample recovery can be adjusted or corrected by addition and use of an internal standard, such as a deuterated internal standard for LC-MS/MS, prior to aspirating and discarding the non-polar phase.

In some embodiments, the separation is physical separation used in combination with magnetic separation. For example, in an aspect, provided is a device (e.g., a magnetized centrifuge or a centrifuge equipped with a magnet that aids in separation by both the gravity and magnetic force of a magnet). In one aspect, provided herein is a device for separation of a particle described herein, the device comprising a magnet and centrifuge. In some embodiments, the device significantly reduces the time of centrifugation.

### Method for the Removal of Interferences

Described herein are methods for removing or minimizing interferences, the method comprising depleting (e.g, mitigating, reducing or managing) known pre-analytical and analytical sources of testing error (e.g., interferences) due to hemolysis, lipemia, icterus, bilirubin, microfibrin clots, cellular debris, blood cells, fibrinogen, other interfering substances such as drugs, metabolites, supplements, herbal remedies, and multivitamins. In some embodiments, the methods described herein provide methods for removing interference due to matrix effects or sample-type differences (e.g., animal species, human species). In some embodiments, the methods described herein provide methods for removing interferences prior to diagnostic testing (e.g., diagnostic or biomarker testing for example in a clinical trial). In some embodiments, the methods for removing biomarkers described herein are used in a clinical trial to improve the accuracy and dependability of a diagnostic test of a biomarker described herein. For example, the methods described herein can be used in patient selection or screening, e.g., for inclusion or exclusion criteria. In some embodiments, the methods or removal or depletion described herein can be used to identify outliers in clinical data or clinical trial results. For example, an outlier in clinical data or clinical trial results include a false positive or false negative identification for a biomarker described herein.

Depletion is defined as complete if sufficient quantity of interference is captured and/or removed for subsequent interference-free or reduced quantitative, semi-quantitative, or qualitative analysis. Depletion is defined as partial if sufficient quantity of interference(s) or interference mechanism(s) is captured and/or removed for subsequent semi-quantitative or qualitative analysis, or also partial if sufficient quantity of interference(s) or interference mechanism(s) and internal standard(s) is captured for quantitative, semi-quantitative or qualitative analysis by measurement methods that can use internal standards to adjust for recovery of the target analyte(s) or biomarker such as LCMS and LC-MS/MS (i.e. deuterated internal standard) and HPLC (C14 or tritiated internal radioisotope internal standards).

Depletion does not imply 100% removal of interference from the sample but means that residual interference no longer results in an erroneous result. However, sample pre-treatment depletion can result in 100% removal of interference if required for a particular assay or purpose such as subsequent elution and analysis by LC-MS/MS, or for sample preanalytical processing, nucleic acid purification and concentration for molecular diagnostics, or for the enrichment of biomarkers from challenging sample types such as urine, saliva and stool.

In some embodiments, the method s described herein is performed is less than 1 week, 6 days, 5 days, 4 days, 3 days, 2 days, 1 day, 24 hours, 20 hours, 16 hours, 12 hours, 8 hours, 4 hours, 2 hours, 1 hour, 30 minutes, 15 minutes, 10 minutes, 5 minutes or less. In some embodiments, the methods described herein is performed in less than 1 day.

### Interferences

The methods provided herein reduce, minimize, or eliminate an interference in a biological sample. Interference is a substance present in a patient specimen that can alter the correct value of the result of a diagnostic test, e.g., by interfering with antibody binding, or that can increase or decrease assay signal by bridging, steric hindrance, or autoantibody mechanisms. “Interferences,” as used herein, refers to any endogenous or exogenous substance or combination of endogenous and/or exogenous substances in blood, plasma, serum, CFS, urine, stool, saliva, semen, amniotic fluid, or other bodily fluids or sample matrices, such as immunoglobulins (IgG, IgM, IgA, IgE, IgD), proteins, antigens, lipids, triglycerides, cellular constituents, foreign substances, chemicals, drugs, drug metabolites, supplements, vitamins, herbal remedies, foreign bodies (viruses, bacterium (gram positive, gram negative), fungi, yeast) and waste products produced by any foreign bodies, food or dietary substances that can interfere with a test and result in an erroneous test results by specific or non-specific interactions with the test raw materials, formulation, biological and synthetic components, test design, and/or test format. Interferences can be, but not limited to, heterophile or heterophile-like interferences such as autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation, such as the chemiluminescent substrate (luminol, isoluminol, isoluminol derivatives, ABEI, ABEI derivatives, ruthenium, acridinium ester), fluorescent labels such as fluorescein or other fluorophores and dyes, capture moieties (streptavidin, neutravidin, avidin, CaptAvidin, polyA, polyDT, aptamers, antibodies, Fab, F(ab′)2, antibody fragments, recombinant proteins, enzymes, proteins, biomolecules, polymers) and their binding partners (i.e. biotin, fluorescein, PolyDT, PolyA, antigen, etc.), conjugation linkers (LC, LC-LC, PEO, PEOn), bovine serum albumin, human serum albumin, ovalbumin, gelatin, purified poly- and monoclonal IgG such as mouse, goat, sheep and rabbit, polyvinyl alcohol (PAA), polyvinylpyrrolidone (PVP), Tween-20, Tween-80, Triton X-100, triblock copolymers such as Pluronic and Tetronic, and commercially available blockers, blocking proteins and polymer-based blocking reagents such those from Surmodics and Scantibodies) typically used in the design of antibody-based diagnostic tests, non-antibody based diagnostic tests, or sample pre-treatment methods and devices for subsequent analysis by mass spectrometry (i.e. HPLC, MS, LC MS, LC-MS/MS), radioimmunoassay (RIA), enzyme-linked immunoassay (ELISA), chemiluminescence immunoassay (CLIA), molecular diagnostics, lateral flow, point-of-care (PoC), CLIA and CLIA waived tests and devices.

In an aspect, provided herein is a method for removing from a biological sample an interference (e.g., biotin), the method comprising providing a particle derivatized with a capture moiety (that will bind to the inference). In some embodiments, the interference is biotin.

In another aspect, a sample can be pre-treated with a particle (e.g., nanoparticle, microparticle) to deplete sex hormone-binding globulin (SHBG) or sex steroid-binding globulin (SSBG) from serum or plasma such that the SH BG-depleted sample could be subsequently tested to measure free or bioavailable hormone or steroid (i.e. free testosterone). In some embodiments, the interference is sex hormone-binding globulin (SH BG) or sex steroid-binding globulin (SSBG).

In some embodiments, the interference is biotin, HAMA, RF, Heterophilic, or anti-SAv.

### Method for the Removal or Enrichment of Biomarkers

Described herein are methods for enriching or increasing the concentration of a biomarker in a biological sample. “Enrichment” is defined as the complete or partial particle capture and binding of target analyte(s) or biomarker to the particles from a biological sample (e.g., human or animal serum, plasma, blood, whole blood, processed blood, urine, saliva, stool (liquid and solid), semen or seminal fluid, cells, tissues, biopsy material, DNA, RNA, or any fluid or solid). In some embodiments, enrichment comprises washing and concentration of a biological sample, for example by allowing the biomarker-specific nanoparticles to be washed, then isolated to remove or minimize interferences prior to a biomarker characterization and measurement step.

In some embodiments, the methods described herein are used to isolate and purify a specific target (e.g., a biomarker) in a biological sample for subsequent elution and testing, or to enrich or increase the concentration of the biomarker prior to the diagnostic test.

After washing or isolating the biomarker specific particles, the particles can be dispersed, reconstituted or resuspended in a buffer such as phosphate buffered saline (i.e. PBS pH 7.2), or LC-MS/MS compatible buffer, prior to the characterization or measurement step. This means the key characterization or measurement step of the captured and enriched biomarkers by the particles occurs in a buffer system and not in the animal or human matrix which is what introduces or causes the matrix effect or bias between biomarkers measured in animal blood, plasma, serum or urine as compared to the same biomarkers measured in blood, plasma, serum or urine using the same characterization, measurement or test method or system. W ash allows sample matrix, components, proteins and cellular constituents and associated interference or matrix effect be washed away.

Enrichment is defined as complete if sufficient quantity of anayte(s) is captured for subsequent diagnostic test, e.g., quantitative, semi-quantitative, or qualitative analysis, and is defined as partial if sufficient quantity of analyte(s) or biomarker is captured for subsequent semi-quantitative or qualitative analysis, or also partial if sufficient quantity of target analyte(s) or biomarker and internal standard(s) is captured for quantitative, semi-quantitative or qualitative analysis by measurement methods that can use internal standards to adjust for recovery of the target analyte(s) or biomarker such as LC MS and LC-MS/MS (i.e. deuterated internal standard) and HPLC (C14 or tritiated internal radioisotope internal standards).

A method is provided herein for enriching a biomarker in a sample prior to a diagnostic test consisting of: a) adding a particle (e.g., nanoparticle, microparticle) to the sample; b) mixing the sample with the particle (e.g., nanoparticle, microparticle); c) incubating the particle (e.g., nanoparticle, microparticle) with the sample to bind and capture the biomarker to the particle (e.g., nanoparticle, microparticle); d) separating or removing the particle (e.g., nanoparticle, microparticle) from the sample; e) saving the particle (e.g., nanoparticle, microparticle); f) washing the particle (e.g., nanoparticle, microparticle) using an appropriate wash diluent to remove non-specific materials; g) measuring the amount, mass, molarity, concentration, or yield of biomarker captured by the particle (e.g., nanoparticle, microparticle) using a qualitative, semi-quantitative or quantitative diagnostic test specific for the biomarker. In some embodiments, the diluent comprises water (e.g., deionized water, water for injection, saline, a buffered aqueous solution).

In some embodiments, the methods of enrichment described herein comprise a washing step. The washing step removes interferences as described herein and/or provides washed, purified, or isolated biomarker of interest (e.g., a biomarker as described herein). In some embodiments, the methods of enrichment described herein reduce matrix effects or species effects. In some embodiments, the methods of enrichment described herein are used prior to a diagnostic test comparing two biological samples of different origin. In some embodiments, the methods of enrichment described herein are used prior to a diagnostic test comparing an animal sample and a human sample. In some embodiments, the methods of enrichment described herein are used prior to a diagnostic test comparing a serum sample and a plasma sample. In some embodiments, the methods of enrichment described herein is used on a sample of high viscosity.

In some embodiments, the methods of enrichment comprise combining of a first biological sample enriched with a biomarker with a second biological sample enriched with the biomarker.

Provided herein is a method of measuring the amount, mass, molarity, concentration, or yield of targeted biomarker captured and enriched by the particle (e.g., nanoparticle, microparticle) whereby the biomarker is eluted, disassociated or freed from the particle (e.g., nanoparticle, microparticle) by the cleavage reagent described herein, e.g., by disrupting the binding interaction using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine-HC, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine-HCO, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

Unless otherwise stated, or implicit from the disclosure, any of the embodiments described in connection with any particular method or composition described herein can be used in conjunction with any of the other embodiments described herein.

The methods and compositions of the invention can be used in conjunction with any suitable assay known in the art, for example any suitable affinity assay or immunoassay known in the art including, but not limited to, protein-protein affinity assays, protein-ligand affinity assays, nucleic acid affinity assays, indirect fluorescent antibody assays (IFA S), enzyme-linked immunosorbant assays (ELISAs), radioimmunoassays (RIAs), and enzyme immunoassays (EIAs), director indirect assays, competitive assays, sandwich assays, CLIA or CLIA waved tests, LC-MS/MS, analytical assays, etc.

A method of both depleting sample interferences and enriching biomarkers from the same sample prior to the diagnostic test consisting of: a) add a chemical and/or biological reagent, additive or composition to the sample to block or deplete sample-specific interferences prior to the addition of a biomarker specific particle (e.g., nanoparticle, microparticle) to the sample; b) add a biomarker specific particle (e.g., nanoparticle, microparticle) to the sample after pretreating or incubating the sample with the chemical and/or biological reagent, additive or composition; c) incubate the biomarker specific particle (e.g., nanoparticle, microparticle) with the sample to bind and capture the targeted biomarker(s) to the particle (e.g., nanoparticle, microparticle); d) wash the particle (e.g., nanoparticle, microparticle) or isolate it from the sample and chemical and/or biological reagent, additive or composition e) characterize the biomarker(s) captured and enriched by the particle (e.g., nanoparticle, microparticle) using a diagnostic test.

For example, in an embodiment, a particle bound to CaptAvidin would bind to biotin in a sample at neutral pH. The biotin bound to the CaptAvidin particle would release biotin when the pH is raised to 10.

### Biomarkers

Described herein are methods to isolate or for isolating or enriching a biomarker present in a biological sample. A “biomarker,” as referred to herein, is defined as a distinctive biological or biologically derived indicator (e.g., a metabolite) of a process, event, or condition such as aging or disease. Biomarkers may be an endogenous and/or exogenous analyte, antigen, small molecule, large molecule, drug, therapeutic agent, metabolite, xenobiotic, chemical, peptide, protein, protein digest, viral antigen, bacteria, cell, cell lysate, cell surface marker, epitope, antibody, a fragment of an antibody, IgG, IgM, IgA, IgE, IgD receptor, a ligand of a receptor, hormone, a receptor of a hormone, enzyme, a substrate of an enzyme, a single stranded oligonucleotide, a single stranded polynucleotide, a double stranded oligonucleotide, a double stranded polynucleotide, polymer and aptamer. In some embodiments, biomarkers is an interference described herein (e.g., a substance present in a patient specimen that can alter the correct value of the result of a diagnostic test, e.g., by interfering with antibody binding, or that can increase or decrease assay signal by bridging, steric hindrance, or autoantibody mechanisms. “Interferences,” as used herein, can be, but not limited to, heterophile or heterophile-like interferences such as autoantibodies, rheumatoid factor (RF), human anti-mouse antibodies (HAMA), human anti-animal antibodies (HAAA) such as goat, rabbit, sheep, bovine, mouse, horse, pig, and donkey polyclonal and/or monoclonal antibodies, and manufacture assay-specific interference used in the test design or assay formulation, such as the chemiluminescent substrate (luminol, isoluminol, isoluminol derivatives, ABEI, ABEI derivatives, ruthenium, acridinium ester), fluorescent labels such as fluorescein or other fluorophores and dyes, capture moieties (streptavidin, neutravidin, avidin, polyA, polyDT, aptamers, antibodies, Fab, F(ab′)2, antibody fragments, recombinant proteins, enzymes, proteins, biomolecules, polymers) and their binding partners (i.e. biotin, fluorescein, PolyDT, PolyA, antigen, etc.), conjugation linkers (LC, LC-LC, PEO, PEOn), bovine serum albumin, human serum albumin, ovalbumin, gelatin, purified poly- and monoclonal IgG such as mouse, goat, sheep and rabbit, polyvinyl alcohol (PAA), polyvinylpyrrolidone (PVP), Tween-20, Tween-80, Triton X-100, triblock copolymers such as Pluronic and Tetronic, and commercially available blockers, blocking proteins and polymer-based blocking reagents such those from Surmodics and Scantibodies) typically used in the design of antibody-based diagnostic tests, non-antibody based diagnostic tests, or sample pre-treatment methods and devices for subsequent analysis by mass spectrometry (i.e. HPLC, MS, LCMS, LC-MS/MS), radioimmunoassay (RIA), enzyme-linked immunoassay (ELISA), chemiluminescence immunoassay (CLIA), molecular diagnostics, lateral flow, point-of-care (PoC), CLIA and CLIA waived tests and devices). In some embodiments, biomarkers are found in biological samples described herein.

Fibrinogen. Fibrinogen is converted during tissue and vascular injury by thrombin to fibrin, which subsequently results in the formation of a fibrin-based blood clot. In some embodiments, the particles described herein (e.g., particle-derivatized anti-fibrinogen (e.g., mouse anti-fibrinogen)) used in the methods described herein bind and allow separation (e.g., chemical separation) of fibrinogen in whole blood. Particle binding to the clot via fibrin can be isolated and removed from the serum post-centrifugation for particle-free serum testing. In some embodiments, the biomarker is fibrinogen. In some embodiments, the methods described herein use particle-derivatized anti-fibrinogen to remove the need for centrifugation of samples (e.g., blood samples).

Traumatic Brain Injury. In one embodiment, the biomarker is for traumatic brain injury. There are nine (9) biomarkers associated with the severity and magnitude of acute brain injury and the integrity of the blood brain barrier (BBB), but they are present at very low circulating concentrations in blood and are very difficult to detect and quantitate using existing immunoassay technologies and test platforms. While the Banyan BTI test (FDA cleared Feb. 14, 2018) measures only 2 of these biomarkers, the methods and devices (e.g., methods of enrichment devices for enrichment) described herein enable simultaneous measurement of all 9 biomarkers in a patient to aid in the near patient diagnosis and prognosis. Particles derivatized with capture moieties for each of the 9 biomarkers may be added to a biological sample from a patient suspected to have TB1. In some embodiments, the traumatic brain injury biomarker is selected from the group consisting of: S100B, GFAP, NLF, NFH, γ-enolase (NSE), α-II spectrin, UCH-L1, total tau, and phosphorylated tau. In some embodiments, the traumatic brain injury biomarker is selected from GFAP and UCH-L1.

In some embodiments, the methods described herein (e.g., methods of enrichment) are used to isolate or enrich the presence of one, two, three, four, five, six, seven, eight, or nine of the traumatic brain injury biomarkers selected from the group consisting of: S100B, GFAP, NLF, NFH, γ-enolase (NSE), α-II spectrin, UCH-L1, total tau, and phosphorylated tau.

Alzheimer's Disease. In one embodiment, the biomarker is for Alzheimer's Disease. There are two (2) biomarkers associated with the severity and magnitude of Alzheimer's Disease. In some embodiments, the Alzheimer's Disease biomarker is selected from the group consisting of: amyloid beta, BACE1, and soluble Aβ precursor protein (sAPP). In some embodiments, the Alzheimer's Disease biomarker is selected from the group consisting of: β-amyloid (1-42), phospho-tau (181p), and total-tau. In some embodiments, the methods described herein (e.g., methods of enrichment) are used to isolate or enrich the presence of one, two or three of the Alzheimer's Disease biomarkers selected from the group consisting of: amyloid beta, BACE1, and soluble Aβ precursor protein (sAPP). In some embodiments, the biomarker is amyloid beta, BACE1, or soluble Aβ precursor protein (sAPP). In some embodiments, the biomarker for Alzheimer's Disease is found in a biological sample (e.g., CSF).

Sexually Transmitted Diseases. In one embodiment, the biomarker is for a sexually transmitted disease (STD). There are at least ten (10) biomarkers characteristic of transmission of a STD. In some embodiments, the STD biomarker is a biomarker for Chlamydia, Gonorrhea, Syphilis, Trichomonas, HPV, Herpes 1 and 2, HSV, Hepatitis A, Hepatitis B, Hepatitis C, HIV 1 and 2. In some embodiments, the methods described herein (e.g., methods of enrichment) are used to isolate or enrich the presence of one, two, three, four, five, six, seven, eight, nine, ten, eleven, twelve or thirteen STD biomarkers: Chlamydia, Gonorrhea, Syphilis, Trichomonas, HPV, Herpes 1 and 2, HSV, Hepatitis A, Hepatitis B, Hepatitis C, HIV 1 and 2, and HIV antibodies. In some embodiments, the biomarker is in urine (e.g., Chlamydia, Gonorrhea, Trichomonas). In some embodiments, the biomarker is in blood, serum, or plasma (e.g., Syphilis, HPV, Herpes 1 and 2, HSV, Hepatitis A, Hepatitis B, Hepatitis C, HIV 1 and 2, HIV antibodies).

Bacterial Infection. In one embodiment, the biomarker is for a bacterial infection, e.g., sepsis. The current gold standard test for bacterial infection is blood culture which can take 24-48 hours before a positive result can be reflexed to a confirmatory test such as molecular diagnostics. Described herein are methods to rule-in/rule-out bacterial infection in as little as 30 minutes or less where time is critical to successfully treat patients to prevent or manage sepsis, for example in 60 minutes or less (e.g., 50 minutes, 40 minutes, 30 minutes, 20 minutes or less). There are at least thirty (30) biomarkers characteristic of bacterial infection. In some embodiments, the bacterial biomarker is selected from the group consisting of a biomarker for sepsis-causing species of bacteria (e.g., Enterococcus faecium, Escherichia coli, Klebsiella pneumoniae, Pseudomonas aeruginosa, and Staphylococcus aureus). In some embodiments, the biomarker is a biomarker for Enterococcus faecium, Escherichia coli, Klebsiella pneumoniae, Pseudomonas aeruginosa, and Staphylococcus aureus. In some embodiments, the biomarker is a biomarker for a gram positive or gram negative bacteria. In some embodiments, the biomarker is a biomarker for a yeast pathogen (e.g., a yeast pathogen associated with bloodstream pathogens).

In some embodiments, the gram positive bacteria is: Enterococcus, Listeria monocytogenes, Staphylococcus, Staphylococcus aureus, Streptococcus, Streptococcus agalactiae, Streptococcus pneumoniae, or Streptococcus pyogenes.

In some embodiments, the gram negative bacteria is: Acinetobacter baumannii, Haemophilus influenza, Neisseria meningitides, Pseudomonas aeruginosa, Enterobacteriaceae, Enterobacter cloacae complex, Escherichia coli, Klebsiella oxytoca, Klebsiella pneumoniae, Proteus, or Serratia marcescens.

In some embodiments, the yeast pathogen is: Candida albicans, Candida glabrata, Candida krusei, Candida parapsilosis, Candida tropicalis.

In some embodiments, followed by mass spectrometry method. Method is envisioned where a cleavage agent (e.g., reducing agent (e.g., DTT or TCEP)) is added to a bacteria-particle bound complex to cleave the linker (i.e., linker conjugating particle to surface capture moiety). The resultant bacterial is grown in culture or analyzed by MALDI-TOF mass spectrometry.

Method is envisioned where a cleavage agent (e.g., reducing agent (e.g., DTT or TCE P)) is added to a bacteria-particle bound complex to cleave the linker (i.e., linker conjugating particle to surface capture moiety). The resultant bacterial is grown in culture or analyzed by MALDI-TOF mass spectrometry or analyzed by a molecular diagnostics such as the FilmArray® Blood Culture Identification (BCID) Panel by BioFire Diagnostics.

Thyroid Function. TSH concentrations are measured as part of a thyroid function test in patients suspected of having an excess (hyperthyroidism) or deficiency (hypothyroidism) of thyroid hormones. The methods described herein in some embodiments are used to evaluate thyroid function. In some embodiments, the biomarker is an antigen (e.g., TSH). In some embodiments, the capture moiety is an autoantibody (e.g., free autoantibody, complexed autoantibody) with specificity to the antigen (e.g., TSH).

In some embodiments, the interference (affecting measurements of thyroid stimulating hormone, free thyroxine, and free triiodothyronine) is macroTSH, biotin, anti-streptavidin antibodies, anti-ruthenium antibodies, thyroid hormone autoantibodies, or heterophilic antibodies.

Cardiac Function. The methods described herein in some embodiments are used to evaluate cardiac function. An increased level of troponin circulating in blood is a biomarker for heart disorders, e.g., myocardial infarction. Cardiac I and T are specific indicators of heart muscle damage. Subunits of troponin are also markers for cardiac health. Specifically cTnI and cTnT are biomarkers for acute myocardial infarction (AMI) for example type 1 and 2 myocardial infarction, unstable angina, post-surgery myocardium trauma and related diseases. In some embodiments, the biomarker is free cTnI, free cTnT, binary cTnI-TnC, or ternary cTnI-TnC-TnT. In some embodiments, the biomarker is an indicator for heart failure. In some embodiments, the biomarker is an indicator for stroke (e.g., as described in https://www.ahajournals.org/doi/10.1161/STROKEAHA.117.017076 and https://www.360dx.com/business-news/roche-test-helps-differentiate-bleeding-risk-stroke-risk-patients-considering #.W1jz0thK hcA, which are incorporated by reference in their entirety). In some embodiments, the biomarker is an indicator for fibrosis (e.g., as described in http://www.onlinejacc.org/content/65/22/2449, which is incorporated by reference in its entirety). In some embodiments, the biomarker is for diagnosis of acute coronary syndrome (ACS). In some embodiments, the biomarker is for Cardiac Troponin (1, I-C, I-C-T, T) and other cardiac troponin fragments, Natriuretic Peptides (BNP, ANP, CNP), N-terminal fragments (i.e. NT-proBNP, NT-proCNP), glycosylated, non-glycosylated, CRP, Myoglobin, Creatinine kinase (CK), CK-MB, sST2, GDF-15, Galectin-3.

In some embodiments, the accuracy and precision by being able to test large sample volumes (i.e. 1 mL, 10 mL, 100 mL, 1000 mL, etc.) to improve likelihood of detection of very dilute or low concentration biomarker(s), as well as very small sample volumes (i.e. neonates, pediatrics, elderly) which typically are untestable today or require sample dilution before testing which compromises test sensitivity, accuracy and precision. In some embodiments, the biological sample is in a 1 mL, 10 mL, 100 mL, 1000 mL or greater volume. In some embodiments, the biological sample is in a 0.5 mL, 0.25 mL, 0.1 mL, 0.05 mL or lesser volume.

Also provided herein is a method for using particle sample pre-treatment to aid in enrichment of biomarkers prior to a diagnostic test by allowing a wash step or particle isolation followed by selective release or elution of the captured biomarker(s), or selective release or elution of the capture moiety-biomarker complex, from the particles prior to the biomarker characterization step or test method.

The use of a “cleavage reagent or “releasing agent” that will disrupt the bond between the capture moiety on the particle surface and the biomarker, e.g., acidic or basic pH, high molarity salt, sugar, chemical displacer, detergent, surfactant, and/or chelating agent, or combination thereof, without displacing or eluting the capture moiety but only the biomarker. After washing or isolating the particles from the sample matrix with magnet(s), the particles can subsequently be treated with an elution solution containing a releasing agent(s) to selectively release the biomarker into solution. The particles can be quickly (less than 2 minutes; ideally less than 30 seconds) isolated to the side(s) and/or bottom of the sample device (vial, test tube, other) to form an essentially particle-free sample supernatant. The particle-free supernatant can be subsequently aspirated without disrupting the pellet comprising particles and dispensed into a separate transfer tube or injected directly onto the analytical system (i.e. LC-MS/MS or MALDI-TOF) for testing of the biomarker.

For example, the cleavage reagent or releasing agent described herein disrupt the binding interaction or cleavable bond as described herein between the particles described herein and a capture moiety described herein, e.g., using elution strategies such as pH (e.g. increased pH with a base such as sodium bicarbonate, decreased pH with an acids such as acetic acid, trichloroacetic acid, sulfosalicylic acid, HCl, formic acid, and common pH elution buffers such as 100 mM glycine-HCl, pH 2.5-3.0, 100 mM citric acid, pH 3.0, 50-100 mM triethylamine or triethanolamine, pH 11.5, 150 mM ammonium hydroxide, pH 10.5), a displacer or displacing agent, competitive elution (e.g. >0.1M counter ligand or analog), ionic strength and/or chaotropic effects (e.g. NaCl, KCl, 3.5-4.0M magnesium chloride pH 7.0 in 10 mM Tris, 5M lithium chloride in 10 mM phosphate buffer pH 7.2, 2.5M sodium iodide pH 7.5, 0.2-3.0M sodium thiocyanate), surfactant, detergent, a concentrated inorganic salt, denaturing (e.g. 2-6M guanidine-HCl, 2-8M urea, 1% deoxycholate, 1% SDS), an organic solvent (e.g. alcohol, chloroform, ethanol, methanol, acetonitrile, hexane, DMSO, 10% dioxane, 50% ethylene glycol pH 8-11.5 (also chaotropic)), radiation or heat (increased temperature), conformational change, disulfide bond reducers (2-mercaptoethanol, dithiothreitol, tris(2-carboxylethyl)phosphine), enzyme inactivation, chaotropic agents (Urea, Guanidinium chloride, Lithium perchlorate), mechanical agitation, sonication, and protein digestive enzymes (pepsin, trypsin), and combinations thereof.

### Method of Characterization

Described herein are methods for depleting and/or enriching biomarkers for subsequent characterization or diagnostic testing. Characterization of a biomarker described herein (e.g., interference) includes identification and/or quantification of a biomarker described herein (e.g., interference described herein).

### Particles of the Invention

Described herein are particles for the isolation, depletion and/or enrichment of biological samples. In some embodiments, the particles comprise a cleavable bond and a capture moiety (e.g., a particle surface functionalized to present one capture moiety. In some embodiments, the particles comprise a non-cleavable bond and a capture moiety (e.g., a particle surface functionalized to present one capture moiety. In some embodiments, the particles described herein comprise a capture moiety (e.g., a capture moiety with high specificity to a biomarker described herein). In some embodiments, the particles described herein (e.g., the surface of the particles described herein, the particle surface not bound to a capture moiety described herein) are inert (e.g. do not exhibit significant binding to a biomarker described herein). In some embodiments, the particles described herein can be used in the diagnostic tests described herein without further modification to the particle or the diagnostic test. In some embodiments, the particles described herein can be added to and removed from a sample without altering the sample (e.g., without adding or removing an additional biomarker (e.g., an interference).

The particles described herein are sufficiently small with a mean diameter from 0.050 micrometers up to 3.00 micrometers, or preferably from 0.100 micrometers to 1.1 micrometers in diameter, or still more preferably 0.200 micrometers to 0.600 micrometers, or even more preferably from 0.100 micrometers to 0.500 micrometers in diameter.

In some embodiments, the particles described herein (e.g., microparticle, nanoparticle) comprise a core or support, wherein the core or support is a paramagnetic or superparamagnetic material selected from the group consisting of iron oxide, ferromagnetic iron oxide, Fe₂O₃, and Fe₃O₄, maghemite, or combinations thereof.

In some embodiments, the particle surface comprises an organic polymer or copolymer, wherein the organic polymer or copolymer is hydrophobic. In some embodiments, the particle (e.g., nanoparticle, microparticle) surface comprises an organic polymer or copolymer such as a material selected from the group consisting of, but not limited to, ceramic, glass, a polymer, a copolymer, a metal, latex, silica, a colloidal metal such as gold, silver, or alloy, polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer such as dextran or poly(ethylene glycol)-dextran (PEG-DEX), or combinations thereof.

As used herein, “blocker” refers to a protein, polymer, surfactant, detergent, or combinations thereof. In some embodiments, the binding of a capture moiety on a particle described herein (e.g., nanoparticle, microparticle) is blocked with a blocker such as a protein, polymer, surfactant, detergent, or combinations thereof. The blocker is selected from the group consisting of a protein such as albumin, bovine serum albumin, human serum albumin, ovalbumin, gelatin, casein, acid hydrolyzed casein, gama globulin, purified IgG, animal serum, polyclonal antibody, and monoclonal antibody, a polymer such as polyvinyl alcohol (PVA) and polyvinylpyrrolidone (PV P), a combination of a protein and polymer, a peptide, a PEGylation reagent such as (PEO)n-NHS or (PEO)n-maleimide, a triblock copolymer such as Pluronic F108, F127, and F68, a non-ionic detergent such as Triton X-100, polysorbate 20 (Tween-20), and Tween 80 (non-ionic), a zwitterionic detergent such CHAPS, a ionic detergent such as sodium dodecyl sulfate (SDS), deoxycholate, cholate, and sarkosyl, a surfactant, a sugar such as sucrose, and a commercial blocker such as Heterophilic Blocking Reagent (Scantibodies), MAK33 (Roche Diagnostics), Immunoglobulin Inhibiting Reagent (IIR) (Bioreclamation), Heteroblock (Omega Biologicals), Blockmaster (JSR), TRU Block (Meridian Life Sciences), and StabilCoat® & StabilGuard® (Surmodics). In some embodiments, the blocker is bound to a particle described herein (e.g., covalently bound, non-covalently bound). In some embodiments, the blocker is not bound (e.g., covalently bound, non-covalently bound) to a particle described herein.

Cleavable bond. In an aspect, capture moiety binds to a biomarker by a cleavable bond described herein. The cleavable bond can be through covalent or non-covalent binding. Examples of non-covalent binding include, affinity, ionic, van der Waals (e.g., dipole/dipole or London forces), hydrogen bonding (e.g., between polynucleotide duplexes), and hydrophobic interactions. Where association is non-covalent, the association between the entities is preferably specific. Non-limiting examples of specific non-covalent associations include the binding interaction between biotin and a biotin-binding protein such as avidin, captavidin, SA, neutravidin, a fragment of SA, a fragment of avidin, a fragment of neutravidin, or mixtures thereof; the binding of a biotinylated Fab, a biotinylated immunoglobulin or fragment thereof, a biotinylated small molecule (such as, for example, a hormone or a ligand of a receptor), a biotinylated polynucleotide, a biotinylated macromolecule (e.g., a protein or a natural or synthetic polymer) to a biotin-binding protein such as avidin, SA, neutravidin, a fragment of SA, a fragment of avidin, a fragment of neutravidin, or mixtures thereof; the binding of a substrate to its enzyme; the binding of a glycoprotein to a lectin specific for the glycoprotein; the binding of a ligand to a receptor specific for the ligand; the binding of an antibody to an antigen against which the antibody is raised; and duplex formation between a polynucleotide and a complementary or substantially complementary polynucleotide; etc.

A cleavable bond, such as a disulfide bond (R-S-S-R) is used to immobilize or bind the capture moiety (i.e. antibody or antibody fragment such as SH-Fab) to the particle. After washing or isolating the particles from the sample matrix, the particles can subsequently be treated with a solution containing a reducing agent such as TC EP or DTT to cleave the disulfide bond and release the capture moiety-biomarker complex into a solution. The particles can be quickly (less than 2 minutes; ideally less than 30 seconds) isolated to the side(s) and/or bottom of the sample device (vial, test tube, other) to form an essentially particle-free sample supernatant. The particle-free supernatant can be subsequently aspirated without disrupting the pellet comprising particles and dispensed into a separate transfer tube or injected directly onto the analytical system (i.e. LC-MS/MS or MALDI-TOF or molecular diagnostics such as the FilmArray Blood Culture Identification Panel) for testing of the capture moiety-biomarker complex.

In some embodiments, the cleavable bond is a disulfide bond (R-S-S-R).

In some embodiments, the cleavable bond is a non-covalent bond between streptavidin or captavidin, avidin, and biotin.

Capture Moieties. Provided herein are particles comprising one capture moiety that bind an interference as described herein, or a biomarker as described here. As referred to herein, “capture moiety” is selected from the group consisting of an antibody, a binding fragment of an antibody, a IgG, a IgM, a IgA, IgE, IgD a receptor, a ligand of a receptor, a hormone, a receptor of a hormone, an enzyme, a substrate of an enzyme, a single stranded oligonucleotide, a single stranded polynucleotide, a double stranded oligonucleotide, a double stranded polynucleotide, an antigen, a peptide, a polymer, an aptamer, and a protein.

In some embodiments, the capture moiety is a protein. A protein can be, for example, a monomer, a dimer, a multimer, or a fusion protein. In specific embodiments, the protein comprises at least one of an albumin such as, for example, antibody, a fragment of an antibody, BSA, ovalbumin, a fragment of BSA, a fragment of ovalbumin, mouse IgG, polymerized mouse IgG, antibody fragments (Fc, Fab, F(ab′)2) and different subclasses (IgG1, IgG2a, IgG2b, IgG3, IgE, IgD) of mouse IgG to target HAMA and RF interference mechanisms, purified animal polyclonal antibodies (i.e. bovine, goat, mouse, rabbit, sheep) to target HAAA interference, streptavidin, ALP, HRP, BSA (conjugated to isoluminol, ruthenium, acridinium) to target M ASI interference or mixtures thereof.

In some embodiments, the capture moiety is a human anti-animal antibody (e.g., mouse IgG, sheep IgG, goat IgG, rabbit IgG, cow IgG, pig IgG, horse IgG). In some embodiments, the capture moiety is a heterophilic antibody (e.g., FR (Fc-specific), Fab, F(ab)′2, polymerized IgG (type 1, 2a, 2b IgG and IgG fragments, serum components). In some embodiments, the capture moiety is an assay specific binder (e.g., biotin, fluorescein, anti-fluorescein poly/M ab, anti-biotin poly/M ab, streptavidin, neutravidin). In some embodiments, the capture moiety is an assay specific signal molecule (e.g., HRP, ALP, acridinium ester, isoluminol/luminol, ruthenium, ABEI/cyclic ABEI). In some embodiments, the capture moiety is an assay specific blocker (e.g., BSA, fish skin gelatin, casein, ovalbumin, PVP, PVA). In some embodiments, the capture moiety is an assay specific conjugate linker (e.g., LC, LC-LC, PEO4, PEO16). In some embodiments, the capture moiety is an antigen autoantibody (e.g., free T3, free T4). In some embodiments, the capture moiety is a protein autoantibody (e.g., MTSH, TnI, TnT, non-cardiac TnT (skeletal muscle disease)). In some embodiments, the capture moiety is a chemiluminescent substrate (e.g., luminol, isoluminol, isoluminol derivatives, ABEI, ABEI derivatives, ruthenium, acridinium ester) or fluorescent label (e.g., fluorescein or other fluorophores and dyes). In some embodiments, the capture moiety is streptavidin, neutravidin, avidin, polyA, polyDT, aptamers, antibodies, Fab, F(ab′)2, antibody fragments, recombinant proteins, enzymes, proteins, biomolecules, polymers, or molecularly imprinted polymers. In some embodiments, the capture moiety is biotin, fluorescein, PolyDT, PolyA, antigen, etc.

In some embodiments, the capture moiety binds biotin (e.g., avidin, streptavidin, neutravidin, CaptAvidin, anti-biotin antibody, antibody fragment, aptimer, molecularly imprinted polymer, etc.)

In at least one embodiment, the present invention provides a binding surface with two or more different capture moieties.

Generation of Capture Moieties. In an aspect, provided is a method for making a capture moiety, the method comprising the production or generation of complex-specific or conformation-specific antibodies to free autoantibodies or autoantibody complexes. Free autoantibodies are autoantibodies that are not already complexed to their antigen target. Complexed autoantibodies are autoantibodies that have formed a complex with their antigen target.

In an aspect, provided is a method for making a capture moiety, the method comprising the production or generation of complex-specific or conformation-specific antibodies to autoantibody complexes like MTSH. In some embodiments, the autoantibody is triiodothyronine (T3) or thyroxine (T4). In some embodiments, the autoantibody complex is MTSH. For example, complex-specific or conformation-specific antibodies can be raised to autoantibody complexes like MTSH, which can be purified from human serum and used as the capture moiety. In this way the antibodies generated would only have specificity to hIgG or hIgM complexes with TSH. MTSH can be purified based on techniques and published methods or by someone skilled in the art of protein biochemistry and purification. In some embodiments, patients with autoimmune disease who have the greatest likelihood of autoantibody assay interference are used to produce or generate autoantibodies. For example, seethe HyTest SES assay for BNP, WO2014114780, WO2016113719 and WO2016113720, the references of which are cited in their entirety.

Thyroid-specific Autoantibodies. For example, in an embodiment, the autoantibody is an anti-thyroid autoantibody (e.g., anti-thyroid peroxidase antibody, thyrotropin receptor antibodies, thyroglobulin antibodies). Anti-thyroid autoantibodies are autoantibodies targeted against one or more components on the thyroid.

In some embodiments, the autoantibody is a free autoantibody (e.g., thyrotropin (TSH).

In some embodiments, the autoantibody is a complexed autoantibody (e.g., MTSH). In some embodiments, the capture moieties described herein are antibodies generated with specificity to complexed autoantibodies or with confirmation specificity to the hIgG and/or hIgM already bound to its antigen target such as MTSH.

Itemized below is a nonlimiting list of substances that may function as one, or alternatively as the other, member of a binding pair consisting of analyte binder (capture moiety) and analyte, depending on the application for which an affinity assay is to be designed. Such substances can be used, for example, as capture moieties (analyte binders) or can be used to generate capture moieties (e.g., by employing them as haptens/antigens to generate specific antibodies) that can be used with the invention. Affinity assays, including immunoassays, can be designed in accordance with the invention to detect the presence and/or level of such substances where they are analytes in a sample. In a specific embodiment, the analyte-binding capture moieties of the invention can be used to detect these substances as analytes in a sample. Alternatively, the substances listed below can be associated with the solid phase support surface in accordance with the invention, and used to capture molecules that interact with them (such as, for example, antibodies or fragments thereof specific for the listed substances, binding proteins, or enzymes).

A nonlimiting list of substances that may function as one, or alternatively as the other, member of a binding pair consisting of analyte binder (capture moiety) and analyte includes: inducible nitric oxide synthase (iNOS), CA 19-9, IL-1α, IL-1β, IL-2, IL-3, IL-4, IL-t, IL-5, IL-7, IL-10, IL-12, IL-13, sIL-2R, sIL-4R, sIL-6R, SIV core antigen, IL-1RA, TNF-α, IFN-gamma, GM-CSF; isoforms of PSA (prostate-specific antigen) such as PSA, pPSA, BPSA, in PSA, non ai-antichymotrypsin-complexed PSA, ai-antichymotrypsin-complexed PSA, prostate kallikreins such as hK2, hK4, and hK15, ek-rhK2, Ala-rhK2, TWT-rhK2, Xa-rhK2, HWT-rhK2, and other kallikreins; HIV-1p24; ferritin, L ferritin, troponin I, BNP, leptin, digoxin, myoglobin, B-type natriuretic peptide or brain natriuretic peptide (BN P), NT-proBNP, CNP, NT-proCNP(1-50), NT-CNP-53(51-81), CNP-22(82-103), CNP-53(51-103), atrial natriuretic peptide (ANP); human growth hormone, bone alkaline phosphatase, human follicle stimulating hormone, human leutinizing hormone, prolactin; human chorionic gonadotrophin (e.g., CGα, CGβ); soluble ST2, thyroglobulin; anti-thyroglobulin; IgE, IgG, IgG1, IgG2, IgG3, IgG4, B. anthracis protective antigen, B. anthracis lethal factor, B. anthracis spore antigen, F. tularensis LPS, S. aureas enterotoxin B, Y. pestis capsular F1 antigen, insulin, alpha fetoprotein (e.g., AFP 300), carcinoembryonic antigen (CEA), CA 15.3 antigen, CA 19.9 antigen, CA 125 antigen, HAV A b, HAV Igm, HBc Ab, HBc Igm, HIV1/2, HBsA g, HBsA b, HCV Ab, anti-p53, histamine; neopterin; s-VCAM-1, serotonin, sFas, sFas ligand, sGM-CSFR, s1CAM-1, thymidinekinase, IgE, EPO, intrinsic factor Ab, haptoglobulin, anti-cardiolipin, anti-dsDNA, anti-Ro, Ro, anti-La, anti-SM, SM, anti-nRNP, antihistone, anti-Sci-70, Sci-70, anti-nuclear antibodies, anti-centromereantibodies, SS-A, SS-B, Sm, U1-RNP, Jo-1, CK, CK-MB, CRP, ischemia modified albumin, HDL, LDL, oxLDL, VLDL, troponin T, troponin I, troponin C, microalbumin, amylase, ALP, ALT, AST, GGT, IgA, IgG, prealbumin, anti-streptolysin, chlamydia, CMV IgG, toxo IgG, toxo IgM, apolipoprotein A, apolipoprotein B, C3, C4, properdin factor B, albumin, ai-acid glycoprotein, α₁-antitrypsin, α₁-microglobulin, α₂-macroglobulin, anti-streptolysin O, antithrombin-III, apolipoprotein Al, apolipoprotein B, β₂-microglobulin, ceruloplasmin, complement C3, complement C4, C-reactive protein, DNase B, ferritin, free kappa light chain, free lambda light chain, haptoglobin, immunoglobulin A, immunoglobulin A (CSF), immunoglobulin E, immunoglobulin G, immunoglobulin G (CSF), immunoglobulin G (urine), immunoglobulin G subclasses, immunoglobulin M, immunoglobulin M (CSF), kappa light chain, lambda light chain, lipoprotein (a), microalbumin, prealbumin, properdin factor B, rheumatoid factor, ferritin, transferrin, transferrin (urine), rubella IgG, thyroglobulin antibody, toxoplasma IgM, toxoplasma IgG, IGF-I, IGF-binding protein (IGFBP)-3, hepsin, pim-1 kinase, E-cadherein, EZH2, and a-methylacyl-CoA racemase, TGF-beta, IL6SR, GAD, IA-2, CD-64, neutrophils CD-64, CD-20, CD-33, CD-52, isoforms of cytochrome P450, s-VCAM-1, sFas, sICAM, hepatitis B surface antigen, thromboplastin, HIV p24, HIV gp41/120, HCV C22, HCV C33, hemoglobin A1c, and GAD65, IA2, vitamin D, 25-OH vitamin D, 1,25(OH)2 vitamin D, 24,25(OH)2 vitamin D, 25,26(OH)2 vitamin D, 3-epimer of vitamin D, FGF-23, sclerostin, procalcitonin, calcitonin, C. dificille toxin A& B, H. pylori, HSV-1, HSV2.

Suitable substances that may function as one, or alternatively as the other, member of a binding pair consisting of analyte binder (capture moiety) and analyte, depending on the application for which an affinity assay is to be designed, and that can be used with the present invention also include moieties, such as for example antibodies or fragments thereof, specific for any of the WHO International Biological Reference Preparations held and, characterized, and/or distributed by the WHO International Laboratories for Biological Standards (available at http:/www.who.int/bloodproducts/re_materials, updated as of Jun. 30, 2005, which lists substances that are well known in the art; the list is herein incorporated by reference).

A partial list of such suitable international reference standards, identified by WHO code in parentheses following the substance, includes: human recombinant thromboplastin (rTF/95), rabbit thromboplastin (RBT/90), thyroid-stimulating antibody (90/672), recombinant human tissue plasminogen activator (98/714), high molecular weight urokinase (87/594), prostate specific antigen (96/668), prostate specific antigen 90:10 (96/700); human plasma protein C (86/622), human plasma protein S (93/590), rheumatoid arthritis serum (W1066), serum amyloid A protein (92/680), streptokinase (00/464), human thrombin (01/580), bovine combined thromboplastin (OBT/79), anti-D positive control intravenous immunoglobulin (02/228), islet cell antibodies (97/550), lipoprotein a (IFCC SRM 2B), human parvovirus B19 DNA (99/800), human plasmin (97/536), human plasminogen-activator inhibitor 1 (92/654), platelet factor 4 (83/505), prekallikrein activator (82/530), human brain CJD control and human brain sporadic CJD preparation 1 and human brain sporadic CJD preparation 2 and human brain variant CJD (none; each cited in WHO TRS ECBS Report No. 926, 53.sup.rd Report, brain homogenate), human serum complement components C1q, C4, C5, factor B, and whole functional complement CH50 (W1032), human serum immunoglobulin E (75/502), human serum immunoglobulins G, A, and M (67/86), human serum proteins albumin, alpha-1-antitrypsin, alpha-2-macroglobulin, ceruloplasmin, complement C3, transferrin (W1031), anti-D negative control intravenous immunoglobulin (02/226), hepatitis A RNA (00/560), hepatitis B surface antigen subtype adw2 genotype A (03/262 and 00/588), hepatitis B viral DNA (97/746), hepatitis C viral RNA (96/798), HIV-1 p24 antigen (90/636), HIV-1 RNA (97/656), HIV-1 RNA genotypes (set of 10l01/466), human fibrinogen concentrate (98/614), human plasma fibrinogen (98/612), raised A2 hemoglobin (89/666), raised F hemoglobin (85/616), hemoglobincyanide (98/708), low molecular weight heparin (85/600 and 90/686), unfractionated heparin (97/578), blood coagulation factor VIII and von Willebrand factor (02/150), human blood coagulation factor VIII concentrate (99/678), human blood coagulation factor XIII plasma (02/206), human blood coagulation factors II, VII, IX, X (99/826), human blood coagulation factors II and X concentrate (98/590), human carcinoembryonic antigen (73/601), human C-reactive protein (85/506), recombinant human ferritin (94/572), apolipoprotein B (SP3-07), beta-2-microglobulin (B2M), human beta-thromboglobulin (83/501), human blood coagulation factor IX concentrate (96/854), human blood coagulation factor IX a concentrate (97/562), human blood coagulation factor V Leiden, human gDNA samples FV wild type, FVL homozygote, FVL heterozygote (03/254, 03/260, 03/248), human blood coagulation factor VII concentrate (97/592), human blood coagulation factor Vila concentrate (89/688), human anti-syphilitic serum (HS), human anti-tetanus immunoglobulin (TE-3), human antithrombin concentrate (96/520), human plasma antithrombin (93/768), human anti-thyroglobulin serum (65/93), anti-toxoplasma serum (TOX M), human anti-toxoplasma serum (IgG) (01/600), human anti-varicella zoster immunoglobulin (W1044), apolipoprotein A-1 (SP1-01), human anti-interferon beta serum (G038-501-572), human anti-measles serum (66/202), anti-nuclear ribonucleoprotein serum (W1063), anti-nuclear-factor (homogeneous) serum (66/233), anti-parvovirus B19 (IgG) serum (91/602), anti-poliovirus serum Types 1,2,3 (66/202), human anti-rabies immunoglobulin (RA 1), human anti-rubella immunoglobulin (RUB1-1-94), anti-smooth muscle serum (W1062), human anti-double-stranded DNA serum (Wo/80), human anti-E complete blood-typing serum (W1005), human anti-echinococcus serum (ECHS), human anti-hepatitis A immunoglobulin (97/646), human anti-hepatitis B immunoglobulin (W1042), human anti-hepatitis E serum (95/584), anti-human platelet antigen-1a (93/710), anti-human platelet antigen-5b (99/666), human anti-interferon alpha serum (B037-501-572), human alphafetoprotein (AFP), ancrod (74/581), human anti-A blood typing serum (W1001), human anti-B blood typing serum (W1002), human anti-C complete blood typing serum (W1004), anti-D (anti-Rh0) complete blood-typing reagent (99/836), human anti-D (anti-Rh0) incomplete blood-typing serum (W1006), and human anti-D immunoglobulin (01/572).

Other examples of suitable substances that may function as one, or alternatively as the other, member of a binding pair consisting of analyte binder (capture moiety) and analyte, depending on the application for which an affinity assay is to be designed include compounds that can be used as haptens to generate antibodies capable of recognizing the compounds, and include but are not limited to, any salts, esters, or ethers, of the following: hormones, including but not limited to progesterone, estrogen, and testosterone, progestins, corticosteroids, and dehydroepiandrosterone, and any non-protein/non-polypeptide antigens that are listed as international reference standards by the WHO. A partial list of such suitable international reference standards, identified by WHO code in parentheses following the substance, includes vitamin B12 (WHO 81.563), folate (WHO 95/528), homocystein, transcobalamins, T4/T3, and other substances disclosed in the WHO catalog of International Biological Reference Preparations (available at the WHO website, for example at page http://www.who.int/bloodproducts/ref_materials/, updated Jun. 30, 2005), which is incorporated herein by reference. The methods and compositions described herein can comprise an aforementioned WHO reference standards or a mixture containing a reference standard.

Other examples of substances that may function as one, or alternatively as the other, member of a binding pair consisting of analyte binder (capture moiety) and analyte, depending on the application for which an affinity assay is to be designed include drugs of abuse. Drugs of abuse include, for example, the following list of drugs and their metabolites (e.g., metabolites present in blood, in urine, and other biological materials), as well any salts, esters, or ethers, thereof: heroin, morphine, hydromorphone, codeine, oxycodone, hydrocodone, fentanyl, demerol, methadone, darvon, stadol, talwin, paregoric, buprenex; stimulants such as, for example, amphetamines, methamphetamine; methylamphetamine, ethylamphetamine, methylphenidate, ephedrine, pseudoephedrine, ephedra, mahuang, methylenedioxyamphetamine (MDS), phentermine, phenylpropanolamine; amiphenazole, bemigride, benzphetamine, bromatan, chlorphentermine, cropropamide, crothetamide, diethylpropion, dimethylamphetamine, doxapram, ethamivan, fencamfamine, meclofenoxate, methylphenidate, nikethamide, pemoline, pentetrazol, phendimetrazine, phenmetrazine, phentermine, phenylpropanolamine, picrotoxine, pipradol, prolintane, strychnine, synephrine, phencyclidine and analogs such as angel dust, PCP, ketamine; depressants such as, for example, barbiturates, gluthethimide, methaqualone, and meprobamate, methohexital, thiamyl, thiopental, amobarbital, pentobarbital, secobarbital, butalbital, butabarbital, talbutal, and aprobarbital, phenobarbital, mephobarbital; benzodiazapenes such as, for example, estazolam, flurazepam, temazepam, triazolam, midazolam, alprazolam, chlordiazepoxide, clorazepate, diazepam, halazepam, lorazepam, oxazepam, prazepam, quazepam, clonazepam, flunitrazepam; GBH drugs such as gamma hydroxyl butyric acid and gamma butyrolactone; glutethimide, methaqualone, meprobamate, carisoprodol, zolpidem, zaleplon; cannabinoid drugs such as tetrahydracannabinol and analogs; cocaine, 3-4 methylenedioxymethamphetamine (MDMA); hallucinogens such as, for example, mescaline and LSD.

### EXAMPLES

### Example 1: Biotin Interference Depletion after High Dose Biotin Ingestion

Endogenous (non-spiked) biotin samples were serially collected. Baseline serum samples were obtained from 5 apparently healthy adult volunteers (4 male, 1 female) by antecubital venous blood draw in BD brand Vacutainer™ 10 mL red top tubes. Each volunteer subsequently ingested a 20 mg dose of biotin (4×5 mg, Finest Nutrition Biotin 5000 mcg Strawberry, Quick Dissolve, Item #938508, distributed by Walgreens). Serum samples were obtained 1, 3, 6, 8, and 24 hours after biotin ingestion. Blood was allowed to clot for 1 hour at RT and centrifuged at 2,000 rpm for 15 min in a Beckman Allegra 6R tabletop centrifuge. For each time point, serum samples from each volunteer was pooled, mixed for 15 min at RT, aliquoted into 1.2 mL aliquots in 2 mL cryo-vials, and frozen at −80° C.

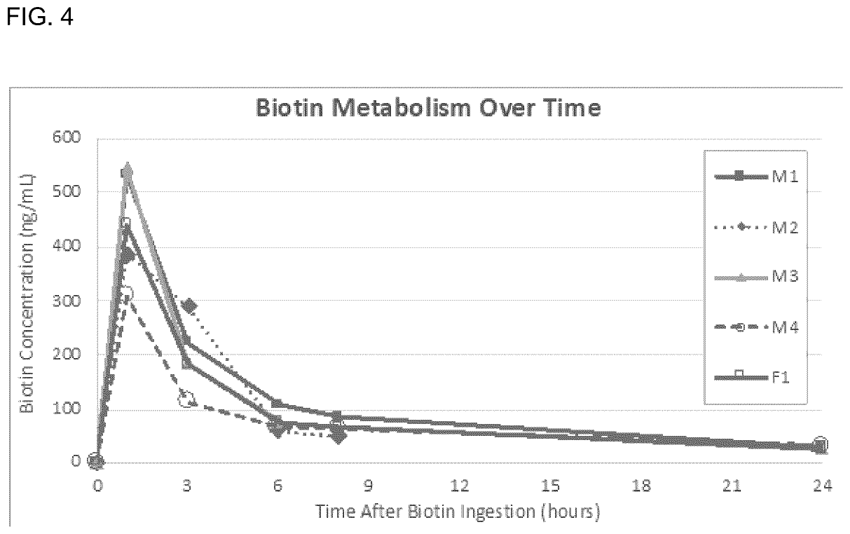

Biotin metabolism was determined by measuring biotin levels in serially collected samples using a free biotin ELISA. The biotin serum samples were tested by the Immundiagnostik IDK® Biotin ELISA kit (Part No. K 8141, Lot No. 180906, measuring range of 48.1-1100 pg/mL). Samples above the kit's measuring range were diluted with the kit's sample dilution buffer. Samples collected 1, 3, 6, and 8 hours after biotin ingestion were posited to be in the 50,000 to 500,000 μg/mL range and were diluted 1:1000. Samples collected 24 hours after biotin ingestion were posited to be near or less than 20,000 μg/mL and diluted 1:20. Samples were tested according to the ELISA kit protocol and biotin levels were still significantly elevated 8 hours (60-107 ng/mL; n=5) and 24 hours (24-32 ng/mL; n=4) after 20 mg biotin ingestion (FIG. 4 and Table 1).

TABLE 1

| | Biotin Concentration (ng/mL) | | | | | |
|---|---|---|---|---|---|---|
| Volunteer | 0-hr | 1-hr | 3-hr | 6-hr | 8-hr | 24-hr |
| M1 | 1.1 | 531 | 221 | 107 | 85 | 29 |
| M2 | 0.1 | 385 | 289 | 60 | 48 | not collected |
| M3 | 0.3 | 546 | 184 | 78 | not collected | 26 |
| M4 | 0.4 | 308 | 113 | 65 | 64 | 32 |
| F1 | 0.4 | 440 | 183 | 76 | 65 | 24 |

High levels of endogenous biotin (370 or 550 ng/mL) were depleted from the serially collected serum samples using superparamagnetic nanoparticles coated with Streptavidin (VERAPREP Biotin reagent) by adding 200 μL serum to a 1.5 mL microcentrifuge tube, adding 20 μL VERAPREP Biotin reagent, gently mixing/rocking the sample for 10 minutes, magnetically separating VERAPREP Biotin reagent for 10 minutes using a Dexter LifeSep® 1.55, carefully aspirating the serum to avoid disrupting the magnetic particles, and by testing the serum sample using a free biotin ELISA.

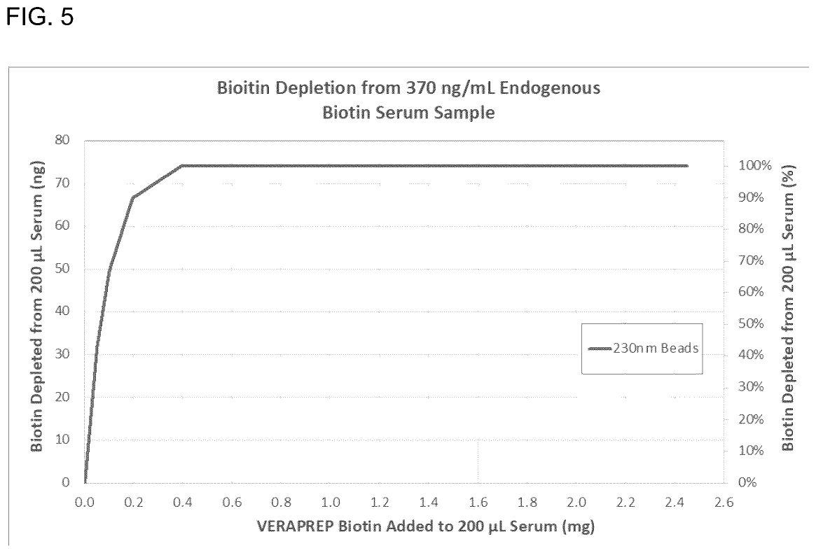

In the first study, increasing amounts (mg) of VERAPREP Biotin reagent was added to different aliquots of the same high biotin (370 ng/mL) endogenous serum sample to determine how much reagent was required to deplete 100% of the free biotin in the sample. 20 μL VERAPREP Biotin reagent (230 nm diameter, 32 μg Streptavidin per mg beads) was added to each 200 μL aliquot of the serum sample collected 1 hour after 20 mg biotin ingestion, mixed by gentle inversion for 10 min at RT, and magnetically separated for 10 min using a Dexter LifeSep 1.55 magnet 175 μL serum supernatant was carefully aspirated and measured by the Immundiagnostik IDK® Biotin ELISA kit (Part No. K8141, Lot No. 180906) and samples above the kit's measuring range were diluted with the kit's sample dilution buffer. The 230 nm VERAPREP Biotin reagent successfully depleted 100% free biotin using a simple 20 min process, 200 μL sample, and only 0.39 mg reagent (FIG. 5)

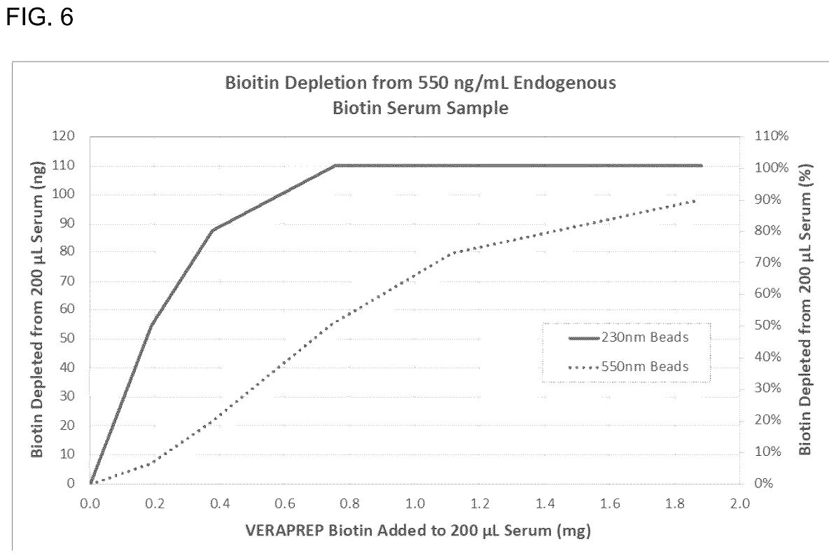

In the second study, increasing amounts (mg) of two different VERAPREP Biotin reagents was added to different aliquots of the same high biotin (550 ng/mL) serum sample to determine how much of each reagent was required to deplete 100% of the free biotin in the sample. 20 μL of 230 nm VERAPREP Biotin reagent (32 μg Streptavidin per mg beads), or 20 μL of 550 nm VERAPREP Biotin reagent (4 μg Streptavidin per mg beads), was added to each 200 μL aliquot of serum collected 1 hour after 50 mg biotin ingestion, mixed by gentle inversion for 10 min at RT, and magnetically separated for 10 min using a Dexter LifeSep 1.55 magnet. 175 μL serum supernatant was carefully aspirated and measured by the Immundiagnostik IDK® Biotin ELISA kit (Part No. K8141, Lot No. 180906) and samples above the kit's measuring range were diluted with the kit's sample dilution buffer. The 230 nm VERAPREP Biotin reagent successfully depleted 100% free biotin using a simple 20 min process, 200 μL sample, and only 0.75 mg reagent, while the 550 nm VERAPREP Biotin reagent only depleted 89% free biotin with 1.86 mg reagent. These results demonstrate that the binding capacity and binding efficiency of VERAPREP Biotin reagent is improved with a smaller bead diameter and increased surface area per unit mass, by increasing the amount of Streptavidin and biotin binding capacity per mg beads, and/or by adding an increased concentration or amount (mg) of the VERAPREP Biotin reagent (FIG. 6).

### Example 2. Biotin Interference Depletion Using an Optimized Sample Pre-Treatment Reagent to Bind and Deplete High Concentrations of Free Biotin in Serum Samples

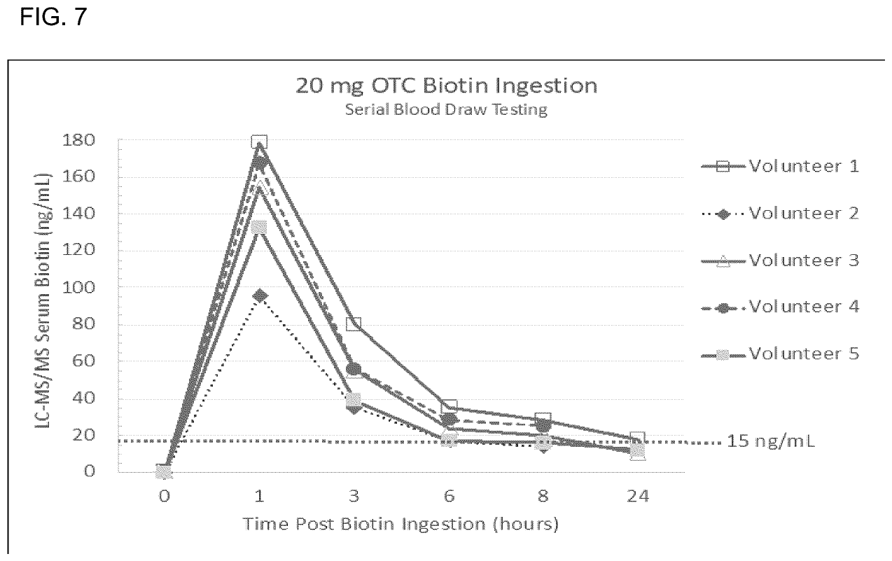

Six volunteers (five apparently healthy adults ages 25 to 46, one Diabetic Type 2 age 65) had fasting serum samples collected at baseline by antecubital venous blood draw in BD brand Vacutainer™ 10 mL red top tubes. Each volunteer subsequently ingested a 20 mg, 100 mg or 200 mg dose of over the counter (OTC) biotin. For the 20 mg dose, serum samples were obtained 1, 3, 6, 8, and 24 hours after biotin ingestion. For the 100 and 200 mg doses, serum samples were collected at 1, 6 and 24 hours after biotin ingestion. Blood was allowed to clot for 1 hour at RT and centrifuged at 2,000 rpm for 15 min in a Beckman Allegra 6R tabletop centrifuge. For each time point and biotin dose, serum samples from each volunteer was pooled, mixed for 15 min at RT, aliquoted into 1.2 mL aliquots in 2 mL cryo-vials, and frozen at −80° C. All samples were sent out for L C-MS/MS biotin measurements at the University of Washington, Department of Laboratory Medicine, 1959 N E Pacific Street, Seattle, WA 98195. For the 20 mg dose, biotin levels were highest at 1 hour [96-179 ng/mL], at 6 hours all 5 volunteers still had serum biotin levels >15 ng/mL [17-35], at 8 hours, 4 out of 5 volunteers had serum biotin levels >15 ng/mL [16-28], and at 24 hours Volunteer 1, a known diabetic Type 2, had a biotin level >15 ng/mL [18](FIG. 7).

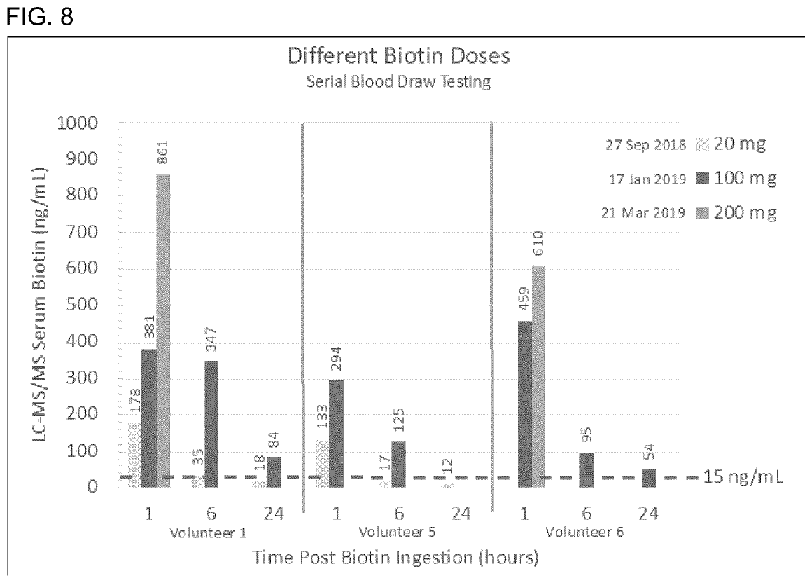

For the 100 and 200 mg doses biotin levels were highest at 1 hour at 294-459 ng/mL for the 100 mg dose, and 610-861 ng/mL for the 200 mg dose. At 6 hours, volunteers who ingested 20 mg or 100 mg biotin had serum biotin levels >15 ng/mL at 17-35 ng/mL for the 20 mg dose, and 95-347 ng/mL for the 200 mg dose. At 24 hours, the 2 volunteers who ingested 100 mg biotin had biotin levels >15 ng/mL with Volunteer 1, known diabetic, at 84 ng/mL, and Volunteer 6 at 54 ng/mL (FIG. 8).

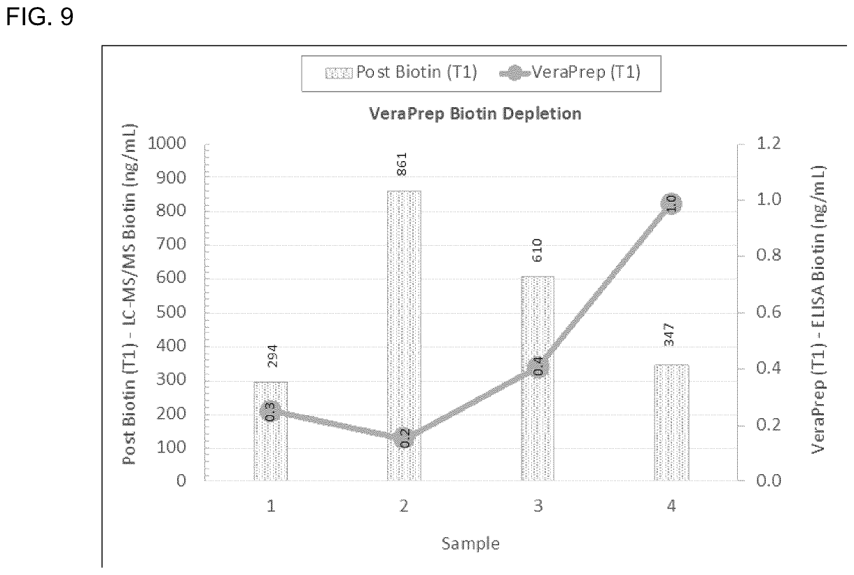

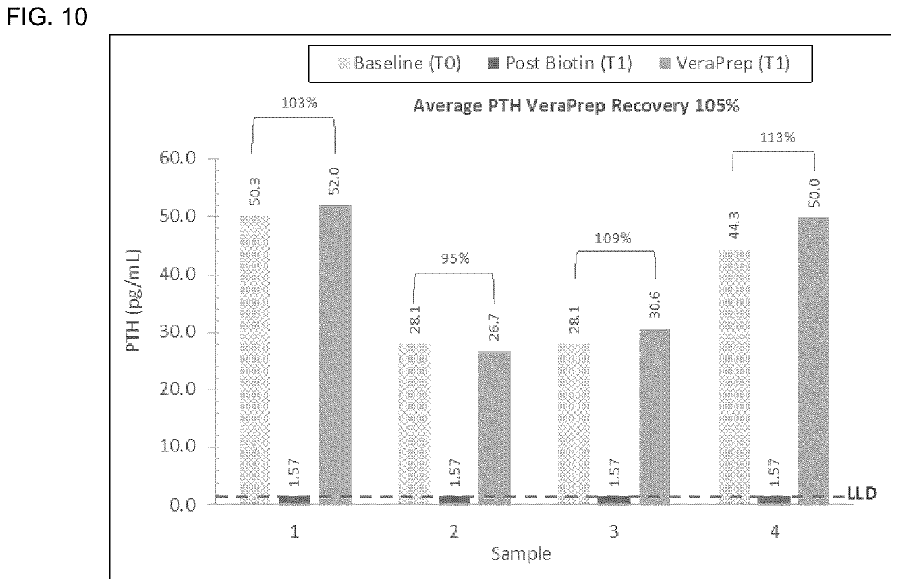

Four samples were selected with high endogenous biotin levels [294-861 ng/mL] measured by LC-MS/MS (FIG. 9). The baseline serum samples were tested by the PTH Intact ELISA (DRG PTH Intact ELISA, Part No. EIA-3645) and PTH values ranged from 28.1 to 50.3 pg/mL. The 1 hour post-biotin ingestion samples all had PTH results were <1.57 pg/mL, or below the lower limit of detection (LLD) (FIG. 10).

All 4 samples were pre-treated with optimized 550 nm superparamagnetic nanoparticles coated with Streptavidin (VeraPrep Biotin), where Samples 1 and 4 had biotin levels <500 ng/mL by L C-MS/MS and were pre-treated with 0.5 mg reagent, and Samples 2 and 3 had biotin levels >500 ng/mL by LC-MS/MS and were pre-treated with 1.5 mg reagent using the following protocol:
1. Remove the VeraPrep Biotin reagent vial from storage and vortex for a minimum of 10 seconds at medium speed to mix well and resuspend the reagent.
2. Insert the reagent vial in the foam vial holder.
3. Insert an empty Micro tube 2 ml (SARSEDT Order Number 72.694) into the LifeSep® 1.5S magnet until the collar of the tube contacts the magnet frame.
4. Dispense 200 μL (0.5 mg) or 600 μL (1.5 mg) of the well-mixed reagent into the empty tube to separate the reagent on the magnet for >30 seconds to form a reagent pellet.
5. Carefully aspirate and discard all of the storage buffer supernatant (˜200 μL or ˜600 μL) without disturbing the reagent pellet.
6. Dispense 400 μL of well-mixed serum or plasma sample into the tube containing the reagent pellet.
7. Tighten the screw the cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

To verify biotin interference removal, VeraPrep Biotin pre-treated samples were tested using the Immundiagnostik IDK® Biotin ELISA kit (Part No. K8141, measuring range of 48.1-1,100 ng/L). Biotin concentrations ranged from 0.2 to 1.0 ng/mL, or within normal plasma levels (200-1,200 ng/L) (FIG. 9). Immediately after VeraPrep Biotin pre-treatment of Samples 1-4, PTH values were measured using the PTH Intact ELISA and PTH values ranged from 26.7 to 52.0 pg/mL (FIG. 10).

The 1 hour post-biotin ingestion samples had high levels of biotin interference per L C-MS/MS (294 to 861 ng/mL) and undetectable PTH values by the PTH Intact E LISA (<1.57 pg/mL), while the 1 hour post-biotin ingestion samples pre-treated with VeraPrep Biotin had physiologically normal biotin values per the Biotin ELISA kit (<1.1 ng/mL) and normal PTH values by the PTH Intact ELISA (26.7 to 52.0 pg/mL)(FIGS. 9 and 10). When comparing VeraPrep Biotin sample pre-treatment PTH values to the baseline PTH values the results recovered from 95 to 113% (mean recovery of 105%)(FIG. 10). A significant difference in test results after VeraPrep Biotin sample pre-treatment, or a significant increase in PTH values in this PTH Intact ELISA sandwich immunoassay, confirms biotin interference was clinically significant in all 4 samples tested.

### Example 3: Low Abundance Biomarker Enrichment

Very low levels of a biomarker in 40 mL PBS, either 0.0195 μIU TSH/mL or 0.497 pg PTH/mL) were enriched using 550 nm superparamagnetic nanoparticles coated with Streptavidin and subsequently coated with biotinylated anti-TSH antibody (VERAPREP Concentrate TSH reagent) or biotinylated anti-PTH monoclonal antibody (VERAPREP Concentrate PTH reagent).

In the first study, VERAPREP Concentrate TSH reagent was prepared by coating 550 nm VERAPREP Biotin with biotinylated anti-TSH capture antibody. 0.08 mL TSH antigen (10 μIU/mL ELISA calibrator) was diluted to 0.0195 μIU/mL in 41 mL PBS buffer below the Functional Sensitivity (<0.054 μIU/mL) of the DRG TSH Ultrasensitive ELISA (Part No. EIA-1790, Lot No. RN58849), and 1 mL was saved as the Baseline Sample (prior to enrichment). The 40 mL sample was processed using a VERAPREP Concentrate TSH protocol to produce a 1.0 mL Enriched Sample for subsequent TSH ELISA testing:

Dilute 80 μL of a 10 μIU/mL TSH standard to 0.0195 μIU/mL in 41.0 mL PBS, and save 1.0 mL as a Baseline Sample (prior to enrichment)
1. Dilute 80 μL of a 10 μIU/mL TSH standard to 0.80 μIU/mL in 1.0 mL VERAPREP Cleave as Control
2. Add 40 mL of 0.0195 μIU/mL TSH in PBS to a 50 mL Falcon Tube
3. Add VERAPREP Concentrate TSH, mix
4. Incubate 60 min at Room Temperature with mixing
5. Magnetically separate VERAPREP Concentrate TSH for 60 min using the Dexter LifeSep® 50SX
6. Decant and discard the 40 mL PBS into waste
7. Add 4.0 mL PBS Wash Buffer, mix
8. Magnetically separate VERAPREP Concentrate TSH in 4 mL PBS Wash Buffer for 30 min using the Dexter LifeSep® 50SX.
9. Decant and discard the 4 mL PBS into waste
10. Add 1 mL PBS Wash Buffer, mix
11. Transfer 1 mL VERAPREP Concentrate TSH to a 1.75 mL conical bottom snap-cap vial
12. Magnetically separate VERAPREP Concentrate TSH in 1 mL PBS Wash Buffer for 10 min using the Dexter LifeSep®1.5S.
13. Aspirate and discard the 1 mL PBS into waste
14. Add 1 mL VERAPREP Cleave, mix
15. Magnetically separate VERAPREP Concentrate TSH in 1 mL VERAPREP C leave for 10 min using the Dexter LifeSep®1.5S.
16. Aspirate and save 1 mL supernatant (Enriched Sample), and test the Control, Baseline Sample and Enriched Sample.

0.08 mL TSH antigen (10 μIU/mL ELI SA calibrator) was also diluted to 0.800 μIU/mL in 1 mL in VERAPREP Cleave buffer as the Control. The Baseline Sample, Enriched Sample, and Control were tested by the DRG TSH Ultrasensitive ELISA, and TSH % Recovery of the Enriched Sample was calculated as[Enriched Sample result]/[Control result]×100%. As expected, the diluted TSH Baseline Sample was undetectable by the Ultrasensitive ELISA and read 0.00 μIU/mL. Using only 0.80 mg reagent, VERAPREP Concentrate TSH successfully enriched the diluted TSH from undetectable to 0.73 μIU/mL (Table 2). As compared to the Control this was a 98.6% recovery, but there may have been a matrix effect of the VERAPREP Cleave buffer in the TSH ELISA that suppressed assay signal (Table 3).

TABLE 2

| | Theorectical TSH | Oberved TSH |
|---|---|---|
| Sample | Result (μlU/mL) | Result (μlU/mL) |
| Control | 0.8000 | 0.74 |
| Baseline Sample | 0.0195 | 0.00 |
| Enriched Sample | 0.7800 | 0.73 |

TABLE 3
% TSH Recovery

([Enriched Sample]/[Control]) × 100%
([0.73 μlU/mL]/[0.74 μlU/mL) × 100% = 98.6%

In the second study, VERAPREP Concentrate PTH reagent was prepared by coating 550 nm VERAPREP Biotin with biotinylated anti-PTH capture antibody. 0.021 mL PTH antigen (971 pg/mL ELISA calibrator) was diluted to 0.497 pg/mL in 41 mL PBS buffer below the Functional Sensitivity (<1.56 pg/mL) of the DRG PTH (Parathyroid) Intact ELISA (Part No. EIA-3645, Lot No. 2896), and 1 mL was saved as the Baseline Sample (prior to enrichment). The 40 mL sample was processed using the VERAPREP Concentrate PTH protocol to produce a 1.0 mL Enriched Sample for subsequent PTH ELISA testing:
1. Dilute 21 μL of a 971 μg/mL PTH standard to 0.497 pg/mL in 41.0 mL PBS, and save 1.0 mL as a Baseline Sample (prior to enrichment)
2. Dilute 21 μL of a 971 μg/mL PTH standard to 20.4 pg/mL in 1.0 mL VERAPREP Cleave as Control
3. Add 40 mL of 0.497 pg/mL PTH in PBS to a 50 mL Falcon Tube
4. Add VERAPREP Concentrate PTH, mix
5. Incubate 30 min at Room Temperature with mixing
6. Magnetically separate VERAPREP Concentrate PTH for 15 min using the Dexter LifeSep® 50SX
7. Decant and discard the 40 mL PBS into waste
8. Add 4.0 mL PBS Wash Buffer, mix
9. Magnetically separate VERAPREP Concentrate PTH in 4 mL PBS Wash Buffer for 10 min using the Dexter LifeSep® 50SX.
10. Decant and discard the 4 mL PBS into waste
11. Add 1 mL PBS Wash Buffer, mix
12. Transfer 1 mL VERAPREP Concentrate PTH to a 1.75 mL conical bottom snap-cap vial
13. Magnetically separate VERAPREP Concentrate PTH in 1 mL PBS Wash Buffer for 10 min using the Dexter LifeSep®1.55.
14. Aspirate and discard the 1 mL PBS into waste
15. Add 1 mL VERAPREP Cleave, mix
16. Magnetically separate VERAPREP Concentrate PTH in 1 mL VERAPREP Cleave for 10 min using the Dexter LifeSep®1.55.
17. Aspirate and save 1 mL supernatant (Enriched Sample), and test the Control, Baseline Sample and Enriched Sample.

0.021 mL PTH antigen (971 pg/mL ELISA calibrator) was also diluted to 20.4 pg/mL in 1 mL in VERAPREP Cleave buffer as the Control. The Baseline Sample, Enriched Sample, and Control were tested by the DRG PTH (Parathyroid) Intact ELISA, and PTH % Recovery of the Enriched Sample was calculated as [Enriched Sample result]/[Control result]×100%. The diluted PTH Baseline Sample read 13.5 pg/mL due to a matrix effect of the VERAPREP Cleave buffer in the ELISA assay. This matrix effect resulted in enhanced assay signal. Using only 0.80 mg reagent, VERAPREP Concentrate PTH successfully enriched the diluted PTH to 42.3 pg/mL (Table 4). As compared to the Control this was a 109% recovery (Table 5).

TABLE 4

| | Theorectical PTH | Oberved PTH |
|---|---|---|
| Sample | Result (pg/mL) | Result (pg/mL)) |
| Control | 20.4 | 38.8 |
| Baseline Sample | 0.497 | 13.5 |
| Enriched Sample | 20.0 | 42.3 |

TABLE 5
% PTH Recovery

([Enriched Sample]/[Control]) × 100%
([42.3 pg/mL]/[38.8 pg/mL) × 100% = 109%

### Example 4: Low Abundance Biomarker Enrichment from Urine for Subsequent Mass Spectrometry (LC-MS/MS or MALDI-MS) Analysis

The following describes a M ass Spectrometry sample pre-treatment protocol to enrich a low abundance biomarker and spiked internal standard (ISTD) from a large volume urine sample using superparamagnetic nanoparticles coated with a capture moiety specific for the biomarker. The exact same protocol could also use a plurality of different superparamagnetic nanoparticles populations mixed together or pooled together, where each population is coated with a different capture moiety, in order to multiplex and enrich more than 1 biomarker and corresponding spiked ISTD from the same sample. The enrichment and characterization of 2 or more biomarkers facilitates the use of an algorithm for the clinical diagnosis and/or prognosis of disease that is not possible with the characterization of a single biomarker. For example, for the diagnosis of obstructive sleep apnea (OSA) from urine, the VERAPREP Concentrate reagent could comprise 4 different antibodies to capture and enrich kallikrein-1, uromodulin, urocortin-3 and orosomucoid-1, or 7 different antibodies to capture and enrich kallikrein-1, uromodulin, urocortin-3 and orosomucoid-1, IL-6, IL-10 and high sensitivity C-reactive protein:
1. Collect patient urine (use standard urine collection protocol such as a urine collection cup)
2. M ix urine collection sample
3. Add 40 mL urine to the 50 mL Falcon Tube
4. Add Deuterated Internal Standard for the biomarker to be enriched, mix
5. Add VERAPREP Condition, mix
6. Add VERAPREP Concentrate, mix
7. Incubate: biomarker+deuterated internal standard capture by VERAPREP Concentrate
8. Magnetically separate VERAPREP Concentrate in the 40 mL urine using the Dexter LifeSep® 50SX
9. Aspirate and discard the urine into waste
10. Add 4 mL PBS Wash Buffer, mix
11. Magnetically separate VERAPREP Concentrate in 4 mL PBS Wash Buffer using the Dexter LifeSep® 50SX.
12. Aspirate and discard the urine into waste
13. Repeat Step 12 two more times (2×)
14. Add 1 mL VERAPREP Cleave, mix (M ass Spectrometry compatible buffer)
15. Magnetically separate VERAPREP Concentrate in 1 mL VERAPREP Cleave using the Dexter LifeSep®1.5S.
16. Aspirate and Test the 1 mL supernatant sample by LC-MS
17. Final biomarker concentration determined based on: 1) 40 mL urine sample size, 2) LC-MS quantitation of the biomarker, and 3) adjust reported biomarker value based on Deuterated Internal Standard recovery

The selective release or cleavage of the captured and enriched biomarker or biomarkers can be accomplished with a change in pH (acidic pH such as glycine pH 2.5 elution followed by neutralization, or alkaline pH 10.0 or greater), using a cleavable linker such as a disulfide bond cleaved with a reducing agent such as TCEP or DTT, or by using competitive elution such a molar excess of D-biotin with monomeric avidin or molar excess of sugar with Concanavalin A that compete for the binding sites on Concanavalin A.

### ABBREVIATIONS

ABEI N-(4-aminobutyl)-N-ethylisoluminol
ALP Alkaline phosphatase
BSA Bovine serum albumin
Fab Fragment antibody-binding
Fc Fragment, crystallizable
HAAA Human anti-animal antibody
HAMA Human anti-mouse antibody
HASA Human anti-sheep antibody
IFU Instructions for use
IgG Antibody or immunoglobulin
IgM Immunoglobulin M
HRP Horse radish peroxidase
LC-MS/MS Liquid chromatography tandem-mass spectrometry
LDT Laboratory developed test
Mab Monoclonal antibody
MASI Manufacture assay specific interference
MFG IVD Manufacturers
PMP Superparamagnetic microparticles
PBCT Primary blood collection tubes
RF Rheumatoid Factor
RLU Relative light units or assay response signal
RUO Research use only
SAv Streptavidin
STT Secondary transfer tubes
TAT Turnaround time
WF Work flow

### Definitions

As used herein, “sample” or “biological sample” refers to any human or animal serum, plasma (i.e. EDTA, lithium heparin, sodium citrate), blood, whole blood, processed blood, urine, saliva, stool (liquid and solid), semen or seminal fluid, amniotic fluid, cerebral spinal fluid, cells, tissues, biopsy material, DNA, RNA, or any fluid, dissolved solid, or processed solid material to be tested for diagnosis, prognosis, screening, risk assessment, risk stratification, and monitoring such as therapeutic drug monitoring. In some embodiments, the sample is a large volume sample. In some embodiments, the sample comprises a plurality of samples (e.g., more than one sample from the same or a different subject. In some embodiments, the sample comprises a biomarker present at low abundance in the sample.

In some embodiments, the sample is collected into in a primary blood collection tube (PBCT), secondary transfer tube (SST), 24-hour (24-hr) urine collection device, vericore tubes, nanotainer, a saliva collection tube, blood spot filter paper, or any collection tube or device such as for stool and seminal fluid, a light green top or green top plasma separator tube (PST) containing sodium heparin, lithium heparin or ammonium heparin, a light blue top tube containing sodium citrate (i.e. 3.2% or 3.8%) or citrate, theophylline, adenosine, dipyridamole (CTAD), a red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

In some embodiments, the sample is a challenging sample type such as urine, 24-hour urine, saliva and stool, or where a biomarker of interest may be dilute or difficult to measure. For example, the biological sample can be a challenging because of the patient population (e.g., neonatal, pediatric, geriatric, pregnant, oncology, autoimmune disease). For example, some biomarkers are too dilute or at too low of concentration, e.g., in circulation, or in urine, to be reliably detected and accurately and precisely measured by existing POCT and central laboratory analyzers. In some embodiments, the challenging sample is cerebrospinal fluid (CSF).

As used herein, “collection device” can be a primary blood collection tube (PBCT), 24-hr urine collection device, a urine collection device, a saliva collection tube, a stool collection device, a seminal fluid collection device, a blood collection bag, or any sample collection tube or device, prior to the addition of the sample.

A PBCT and secondary transfer tube (SST) can be any commercially available standard or custom collection tube (with or without gel separators) from companies like Becton Dickinson (BD), Greiner, VW R, and Sigma Aldrich, a glass tube, a plastic tube, a light green top or green top plasma separator tube (PST) containing sodium heparin, lithium heparin or ammonium heparin, light blue top tube containing sodium citrate (i.e. 3.2% or 3.8%) or citrate, theophylline, adenosine, dipyridamole (CTAD), red top tube for Serology or Immunohematology for the collection of serum in a glass (no additives) or plastic tube (contains clot activators), a red top tube for Chemistry for the collection of serum in a glass (no additives) or plastic tube (contains clot activators), a purple lavender top tube containing EDTA K2, EDTA K3, liquid EDTA solution (i.e. 8%), or EDTA K2/gel tubes for testing plasma in molecular diagnostics and viral load detection, a pink top tube for Blood Bank EDTA, a gray top tube containing potassium oxalate and sodium fluoride, sodium fluoride/EDTA, or sodium fluoride (no anticoagulant, will result in a serum sample), a yellow top tube containing ACD solution A or ACD solution B, a royal blue top (serum, no additive or sodium heparin), a white top tube, or any color or tube type, for any application or diagnostic test type, containing no additives or any additive or combinations thereof, for the collection of blood.

As used herein, a “storage device” or “transfer device” refers to a device that receives the sample and/or other components received in a collection device. The storage or transfer device can be a plastic or glass tube, vial, bottle, beaker, flask, bag, can, microtiter plate, ELISA plate, 96-well plate, 384-well plate 1536 well plate, cuvette, reaction module, reservoir, or any container suitable to hold, store or process a liquid sample.

As referred to herein, a “diagnostic test” includes, but is not limited to any antibody-based diagnostic test, non-antibody based diagnostic test, a sample pre-treatment method or device for subsequent analysis by chromatographic, spectrophotometric, and mass spectrometry methods (i.e. HPLC, MS, LCMS, LC-MS/MS) such as immunoextraction (IE) and solid phase extraction (SPE), radioimmunoassay (RIA), enzyme-linked immunoassay (ELISA), chemiluminescence immunoassay (CLIA), molecular diagnostics, lateral flow (LF), point-of-care (PoC), direct to consumer (DTC), CLIA and CLIA waived tests and devices, Research Use Only (RUO) test, In Vitro Diagnostics (IVD) test, Laboratory Developed Test (LDT), companion diagnostic, and any test for diagnosis, prognosis, screening, risk assessment, risk stratification, and monitoring such as therapeutic drug monitoring. In some embodiments, the diagnostic test comprises short turn-around time (STAT) diagnostic tests, ambulatory tests, lateral flow tests, point of care (PoC) tests, molecular diagnostic tests, HPLC, MS, LCMS, LC-MS/MS, radioimmunoassay (RIA), enzyme-linked immunoassay (ELISA), chemiluminescence immunoassay (CLIA), CLIA and CLIA waived tests, and any diagnostic test used for the diagnosis, prognosis, screening, risk assessment, risk stratification, treatment monitoring, and therapeutic drug monitoring.

1.-50. (canceled)

51. A method for reducing the amount of an interference in a biological sample, the method comprising:
a) combining the sample with a particle comprising an interference capture moiety to provide a mixture;
b) mixing the mixture to provide particle complexes to the interference; and
c) removing the particle complexes to provide a depleted solution;
thereby reducing the amount of the interference.

52. The method of claim 51, wherein said biological sample comprises blood, plasma, serum, cerebrospinal fluid, urine, stool, saliva, semen, or amniotic fluid.

53. The method of claim 51, wherein the sample volume is 1 mL, 10 mL, 100 mL, or 1000 mL.

54. The method of claim 51, wherein the sample volume is 0.5 mL, 0.25 mL, 0.1 mL, or 0.05 mL.

55. The method of claim 51 wherein said particle is from 0.05 μm to 3 μm in diameter.

56. The method of claim 55, wherein said particle comprises a paramagnetic or superparamagnetic material selected from iron oxide, ferromagnetic iron oxide, Fe₂O₃, and Fe₃O₄, maghemite, and combinations thereof.

57. The method of claim 55, wherein the particle surface comprises an organic polymer or copolymer selected from the group consisting of ceramic, glass, a polymer, a copolymer, a metal, latex, silica, gold, silver, an alloy, polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer, dextran, poly(ethylene glycol)-dextran (PEG-DEX), and combinations thereof.

58. The method of claim 51, wherein the method further comprises subjecting the depleted solution to characterization.

59. The method of claim 51, wherein the particle is provided as a lyophilized product.

60. The method of claim 51 wherein said interference comprises human anti-mouse antibodies, rheumatoid factor (RF), anti-goat IgG antibodies, anti-sheep IgG antibodies, anti-rabbit IgG antibodies, anti-cow IgG antibodies, non-specific autoantibodies against human IgG, non-specific autoantibodies against human IgA, non-specific autoantibodies against human IgM, autoantibodies against streptavidin, autoantibodies against biotin, autoantibodies against polyhistidine tags, autoantibodies against bovine serum albumin, or autoantibodies against polyethylene glycol.

61. A method for increasing the accuracy of a diagnostic test by removing an interference from a biological sample, the method comprising:
a) pretreating the biological sample by combining it with a particle comprising an interference capture moiety to provide a mixture;
b) mixing the mixture to provide particle complexes to the interference;
c) removing or eliminating the particle complexes to provide a depleted solution; and
d) subjecting the depleted solution to the diagnostic test, wherein the accuracy of the diagnostic test is increased as compared to performance of the diagnostic test without pretreatment of the sample.

62. The method of claim 61 wherein at least 1%, 3%, 5%, 10%, 20%, 30%, 40%, 50%, 60%, 70%, 80%, 90%, 99% of the interference is removed in comparison to a biological sample not pretreated.

63. The method of claim 62, wherein a sufficient amount of interference is removed to provide less than 100 ppm interference in the biological sample.

64. The method of claim 63, wherein a sufficient amount of interference is removed to provide a less than detectable amount of the interference in a diagnostic test.

65. The method of claim 61 wherein said interference comprises human anti-mouse antibodies, rheumatoid factor (RF), anti-goat IgG antibodies, anti-sheep IgG antibodies, anti-rabbit IgG antibodies, anti-cow IgG antibodies, non-specific autoantibodies against human IgG, non-specific autoantibodies against human IgA, non-specific autoantibodies against human IgM, autoantibodies against streptavidin, autoantibodies against biotin, autoantibodies against polyhistidine tags, autoantibodies against bovine serum albumin, or autoantibodies against polyethylene glycol.

66. The method of claim 61, wherein said biological sample comprises blood, plasma, serum, cerebrospinal fluid, urine, stool, saliva, semen, or amniotic fluid.

67. The method of claim 61, wherein said particle is from 0.05 μm to 3 μm in diameter.

68. The method of claim 67, wherein said particle comprises a paramagnetic or superparamagnetic material selected from iron oxide, ferromagnetic iron oxide, Fe₂O₃, and Fe₃O₄, maghemite, and combinations thereof.

69. The method of claim 68, wherein the particle surface comprises an organic polymer or copolymer selected from the group consisting of ceramic, glass, a polymer, a copolymer, a metal, latex, silica, gold, silver, an alloy, polystyrene, derivatized polystyrene, poly(divinylbenzene), styrene-acylate copolymer, styrene-butadiene copolymer, styrene-divinylbenzene copolymer, poly(styrene-oxyethylene), polymethyl methacrylate, polymethacrylate, polyurethane, polyglutaraldehyde, polyethylene imine, polyvinylpyrrolidone, polyvinyl alcohol, polyacrylic acid, N,N′-methylene bis-acrylamide, polyolefeins, polyethylene, polypropylene, polyvinylchloride, polyacrylonitrile, polysulfone, poly(ether sulfone), pyrolized materials, block copolymers, and copolymers of the foregoing, silicones, or silica, methylol melamine, a biodegradable polymer, dextran, poly(ethylene glycol)-dextran (PEG-DEX), and combinations thereof.

70. The method of claim 61, wherein said interference capture moiety comprises animal antibodies such as mouse, rabbit, goat, sheep, cow antibodies, an assay specific binder, an assay specific signal molecule, an assay specific conjugate linker, antigens recognized by autoantibodies, a chemiluminescent substrate, streptavidin, neutravidin, avidin, polyA, polyDT, an aptamers, Fab, F(ab′)2, an antibody fragment, an enzyme, a protein, or combinations thereof.
